(19) United States
(12) Patent Application Publication
(10) Pub. No.: US 2005/0059038 A1
(43) Pub. Date: Mar. 17, 2005

(54) Method of screening for target ligands

(76) Inventors: Steven P. Adams, Andover, MA (US); Daniel L. Flynn, Lawrence, KS (US); Michael G. Kelly, South San Francisco, CA (US); Gergely M. Makara, Norwood, MA (US); Keith A. Mason, Brighton, MA (US); Ciamac C. Moallemi, Stanford, CA (US); Huw M. Nash, Cambridge, MA (US); Edward A. Wintner, Seattle, WA (US); Zhongli Zheng, Lexington, MA (US)

Correspondence Address: FISH & RICHARDSON PC, 225 FRANKLIN ST, BOSTON, MA, 02110, US

(21) Appl. No.: 10/806,758

(22) Filed: Mar. 22, 2004

Related U.S. Application Data
(60) Provisional application No. 60/456,816, filed on Mar. 21, 2003.
(60) Provisional application No. 60/456,901, filed on Mar. 21, 2003.

(51) Int. Cl. C12Q 1/68; C12Q 1/37; G01N 33/00

(52) U.S. Cl. 435/6; 435/23; 436/86

(57) ABSTRACT

A general system for screening targets (e.g., biomolecules) is described. The screening can be used to discover compounds that bind to a naturally occurring low activity or inactive state of a target and act as inhibitors of the target function as seen in subsequent biological assays.

### CROSS-REFERENCE TO RELATED APPLICATIONS

This application claims priority to U.S. Provisional Application Ser. No. 60/456,816, filed on Mar. 21, 2003, and U.S. Provisional Application Ser. No. 60/456,901, filed on Mar. 21, 2003, the contents of each of which is incorporated by reference herein in its entirety.

### BACKGROUND

The effort to discover small molecule drugs to treat various diseases has been revolutionized with the elucidation of the human genome. High throughput technologies in both genomics and biological screening, together with combinatorial chemistry and computer technology, have made it more feasible to perform massive searches for new drug candidates.

Scientists must sift through enormous numbers of potential drug candidates in their search for a single, effective drug. While combinatorial chemistry and parallel synthesis provide a large number of compounds as potential screening pools, the current screening processes are now a limitation in finding drug leads with novel mechanisms. Most current screening processes are function-based requiring a biochemical or cellular reporter read-out. For enzymes, the reporter can be the consumption of substrate or the generation of product, and for optimal performance the most active form of the target enzyme is desired to configure the function based assays. Function-based screening processes are biased towards identifying only those compounds that directly interfere with the activity of the active form of an enzyme. Therefore, enzymes that are in an inactive form such as an unactivated form or a basal form, and pro-enzyme forms will not be suitable for use in a function-based screen. Nonetheless, a compound binding to the unactivated form and preventing it from being activated can be a very novel and useful drug lead which might be overlooked using many current screening processes. Additionally, if an enzyme requires cofactors for enzymatic activity, functional-based screening processes cannot be configured optimally to find compounds that may bind to an allosteric site existing only when the enzyme is in a cofactor-free form.

Another situation where traditional screening methods may overlook potential drug candidates is that wherein the ligand receptor process involves multimerization of a monomeric protein. A function-based screening process will not be able to detect compounds binding the monomeric form; however, a compound binding to the monomeric form and preventing the multimerization process can be a very useful drug lead. In the case of receptor proteins such as G-protein coupled receptor (GPCR) and nuclear-hormone receptor (NHR), for example, function-based screening for antagonists require at least one known agonist as a reporter. Therefore, function-based screening of orphan (or unliganded) receptors can only discover compounds that stimulate activity of the receptors. Even so, a compound bound to the receptor, but not interfering with ligand binding to the receptor, can still affect the functions of the receptor through interaction with other biomolecules that play a role in the receptor's function. Obviously, the orphan receptors cannot be configured in a function-based screen. Similarly, a protein of unknown function cannot be the target for a function-based screen. Many new proteins of unknown function are discovered through genomic and proteomic experimentation. Thus, there is a need for a function-neutral screening process for the discovery of compounds that bind to all forms of a target. Affinity-based screening processes reported herein can be configured to be function-neutral.

### SUMMARY

The invention relates in part to a general system for screening targets (e.g., biomolecules) in a novel fashion to discover compounds that bind to a naturally occurring low activity or inactive state of a target and act as inhibitors of the target function as seen in subsequent biological assays. This system can be applied to all types of biological targets including DNA, RNA, proteins (e.g., membrane-associated proteins, enzymes, nuclear hormone receptors, and G-protein coupled receptors (GPCRs)). Additionally, the system does not necessitate a biochemical assay for its output and utilizes only very small quantities of a purified protein or other biomolecule receptor, typically less than 1 μg per experiment. What's more, this system does not require knowledge of the receptor's structure for its implementation. Further, the desired output of the methods described herein can be achieved with mixtures of compounds. Also, multiple forms of a given target can be multiplexed together to expedite the efficiency of the process.

In one aspect, the invention provides for an affinity screening method of screening a mixture of compounds (e.g., a mixture having between 2 and 25,000, between about 5 and 10,000, between about 5 and 5,000, between about 5 and 1,000, between about 20 and 500, or between about 100 and 300 compounds) against an unactivated or inactive form of a target moiety. The screening occurs under conditions where the unactivated or inactive form of a target moiety predominates within the reaction. In another aspect, the screening occurs in the absence of or devoid of any one or more components that can activate the target, for example, in the absence of substrate (e.g., ATP, GTP), cofactor(s), metal ions. In another aspect the target is modified or mutated such that it is incapable of being activated (e.g., a protease mutated such that it cannot cleave itself). In another aspect, the target mixture is absent any highly active or physiologically active target. For all of these aspects, the method includes the following steps:
providing a mixture of compounds;
incubating the mixture with the unactivated form of the target moiety to form compound:target complexes;
separating compound:target complexes from unbound compounds and targets;
dissociating compound:target complexes;
identifying the dissociated compounds which had bound to the target by passing the compound through a mass spectrometer, wherein the identified compounds bind to the target moiety.

In one embodiment, the identified compounds can bind to the target moiety with an affinity of Kd between 1 pM (picomolar) and 50 uM (micromolar). The compound mixture can be mass-coded as a means to ensure that at least 90% of the compounds having a unique ion mass is detectable by a mass spectrometry. The target can be any biomolecule such as for example nucleic acid (e.g., DNA or RNA) or enzyme (e.g., kinase, synthase, phosphatase, methylase).

In another aspect, the invention provides for an affinity screening method for screening a mixture of compounds (e.g., a mixture having between 2 and 25,000, between about 5 and 10,000, between about 5 and 5,000, between about 5 and 1,000, between about 20 and 500, or between about 100 and 300 compounds) against a mixture of different forms of a target moiety. In another aspect, the unactivated or inactive form predominates within the target mixture. In another aspect, the reaction is devoid or absent any activating component, for example, in the absence of substrate (e.g., ATP, GTP), cofactor(s), metal ions. In these aspects, the method includes the following steps:
providing a mixture of compounds;
providing a mixture of active forms (e.g., ligand-bound forms) and unactive or inactive forms (e.g., ligand-free forms) of a target moiety;
incubating the mixture of compounds with the mixture of the target moiety to form compound:target complexes;
separating compound:target complexes from unbound compounds and target moieties;
dissociating compound:target complexes;
identifying the dissociated compounds which had bound to the target by passing the compound through a mass spectrometer, wherein the identified compounds bind to the target moiety with an affinity of Kd between 1 pM and 50 uM.
identifying new ligands from among the compounds present in compound:target complexes bound to any form of the target moiety by passing compound:target complexes through a mass spectrometer to identify ligands bound to any form of the target moiety, wherein the identified ligands bind to the target moiety with an affinity of Kd between 1 pM and 50 uM; and
the method can optionally include the step of incubating the new ligands identified with ligand-bound and ligand-free forms of the target moiety, wherein the steps of formation of compound:target complexes and the identification of compounds which are ligands are repeated to delineate which compounds bind to the ligand-bound form versus which compounds bind to the ligand-free form of the target moiety.

In one embodiment, the compound mixture is mass-coded to ensure at least 90% of the compounds have a unique ion mass detectable by mass spectrometry. In another embodiment, the mixture of target forms includes unactivated forms (e.g., unphosphorylated, phosphorylated, unliganded, bound to a negative regulator), inactive forms (e.g., mutated and truncated), and active forms (e.g., phosphorylated, unphosphorylated, bound to agonist, constitutively activated) of a biomolecule. In another embodiment, the mixture of target forms includes monomeric forms and multimeric forms. In another embodiment, the mixture of target forms includes ligand-bound forms and ligand-free forms. In yet another embodiment, the mixture of target forms includes cofactor-bound forms and cofactor-free forms.

In another embodiment, the mixture of compounds is mass-coded and the mixture of target forms includes unactivated forms, inactive forms, active forms, multimeric forms, monomeric forms, ligand-bound forms, ligand-free forms, cofactor-bound forms, and/or cofactor-free forms. The mixture of compounds is mass-coded in such a way as to ensure that at least 90% of the compounds have a unique ion mass detectable by mass spectrometry.

The present invention also provides a method for discovering ligands of kinases. These ligands are selective for the kinase of interest and can serve to inhibit the kinase. Selective kinase inhibitors can be identified by screening kinases (i.e., the target) in their basal or unactivated form (e.g., unphosphorylated, undimerized, or phosphorylated). Kinases screened under these conditions preferentially target an allosteric site formed when the DFG is in the DFG-out position. Screening is performed using an affinity method under conditions where the allosteric site predominates. For example, the allosteric site may predominate when kinases are not catalytically active (e.g., are unactive, in a basal low activity state, or inactive). The invention provides the additional advantage of allowing the screening of large compound mixtures by a method that allows for direct detection and structural assignment of inhibitors. The invention also allows the rationale design of scaffolds, compounds, and libraries based on modeling of the allosteric site formed when the kinase is in the DFG-out conformation. Inhibitors derived by the present invention preferentially target the allosteric site and typically exhibit improved selectivity.

In one aspect the invention provides a method of identifying or discovering a test kinase inhibitor from a library or mixture of compounds (e.g., a mass-coded library). The method generally includes contacting or incubating members of the library or mixture of compounds with a test kinase (e.g., a kinase in the unactivated state). The bound compounds are then separated from the unbound compounds. In another embodiment, the bound compounds are then optionally separated from the unbound compounds. Bound compounds (i.e., potential inhibitors) can then be identified (e.g., by mass-spectrometry). The method can further include determining whether the potential binder inhibits or decreases activity of the test kinase (e.g., in a conventional assay, e.g., biochemical or cell-based assay) including the steps of contacting the inhibitor to the test kinase under conditions and for a time sufficient to allow the inhibitor to bind to the test kinase; and determining if the test kinase is rendered non-functional or less functional by the inhibitor. In one embodiment, incubating or contacting the mixture of compounds (e.g., mass-coded library) is performed in the absence of ATP and/or peptide substrate. In another embodiment, incubating or contacting the mixture of compounds (e.g., mass-coded library) is performed in the presence of ATP and/or peptide substrate. In one embodiment the test kinase is a full-length kinase. In another embodiment, the test kinase includes a truncated fragment of the full-length kinase, that contains a catalytic domain. In another embodiment, the test kinase is a kinase variant or mutant. In another embodiment, the test kinase is unactivated. In another embodiment, the test kinase is activated. In another embodiment, the test kinase is partially active relative to its physiologically active state. In another embodiment, the test kinase is in a basal form, exhibiting low catalytic activity. In another embodiment, identification of the compounds that can bind the test kinase can be identified by mass-spectrometry which can be performed by comparison to a database of mass-coded compounds.

The invention provides for a method of identifying a kinase inhibitor by using the positioning of the DFG motif in the DFG-out conformation, such that a concave pocket is formed as an inhibitor binding site if the DFG motif is in the DFG-out conformation. In one aspect, the invention provides a method of designing an inhibitor. The method includes the steps: a) comparing an unactivated test kinase to a reference kinase whose 3-dimensional structure is known (e.g., Protein Data Bank identifiers: lkyl chain a, 1kv2 chain a, 1iep chain a, 1iep chain b, 1fpu chain a, 1fpu chain b, and 1irk; the Protein Data Bank can be found at the URL address, rcsb.org/pdb/); b) identifying an allosteric binding site (e.g., an allosteric binding site located relative to the DFG motif and the helix α-C); and c) designing an inhibitor based on the allosteric binding site. Designing the inhibitor can include designing a scaffold based on topological and electronic properties of the allosteric binding site and designing a mass-coded library based on the scaffold. In another embodiment, step c) comprises providing a mass-coded library or mixture of compounds. In another embodiment, the method further includes screening (e.g., affinity screening with the test kinase) the mass-coded library for compounds that bind the test kinase and determining whether the kinase binder inhibits the kinase by performing a kinase assay in the presence and absence of the inhibitor, thereby designing or discovering an inhibitor of the test kinase.

In one embodiment, the reference kinase whose 3-dimensional structure is known is a kinase with Protein Data Bank identifier 1g3n chain a, 1g3n chain b, lkyl chain a, 1kv2, chain a, 1iep chain a, 1iep chain b, 1fpu chain a, and 1fpu chain b, or 1irk. In another embodiment, the test is a kinase with Protein Data Bank identifier 1g3n chain a, 1g3n chain b, lkyl chain a, 1kv2, chain a, 1iep chain a, 1iep chain b, 1fpu chain a, and 1fpu chain b, or 1irk.

In another embodiment, the allosteric binding site is identified by locating the DFG motif and determining that the DFG motif is in the DFG-out position. The allosteric site can be located relative to the DFG motif in the DFG-out conformation and relative to the helix α-C. In another embodiment, the DFG motif in the DFG-out position is greater than 11 Å and less than 20 Å in distance from the alpha helix C. In another embodiment, the alpha helix C is analogous in relative 3-dimensional location to insulin receptor kinase alpha helix C containing Val1050-Met1051. The alpha helix C can be homologous in relative 3-dimensional location to the c-abl alpha helix C containing Val289-Met290. The DFG motif can have the sequence DWG or DLG. In one embodiment, the test kinase is c-abl, p38MAPK, or insulin receptor tyrosine kinase. In another embodiment, the test kinase is not c-abl, p38MAPK, or insulin receptor tyrosine kinase. In another embodiment, the allosteric binding site is spatially distinct from the ATP binding site and the activation loop.

In another aspect, the invention provides a method of identifying an inhibitor of a test kinase from a mixture of compounds (e.g., a library, e.g., a mass-coded library) which has been designed based on the topology and electrostatic properties of the allosteric binding site (e.g., concave pocket) which is formed when the DFG is in the DFG-out conformation in a kinase (e.g., an unactivated kinase). The method generally includes producing a mass-coded set of chemical compounds having the general formula A(B)ₙ, where A is a scaffold, each B is, independently, a peripheral moiety, and n is an integer greater than 1, typically from 2 to about 6. The method comprises selecting a peripheral moiety precursor subset from a peripheral moiety precursor set, which is based on the topology and electrostatic properties of the kinase (e.g., unactivated kinase) allosteric site formed with DFG in the DFG-out conformation. The subset includes a sufficient number of peripheral moiety precursors that at least about 50, 100, 250 or 500 distinct combinations of n peripheral moieties derived from the peripheral moiety precursors in the subset. The subset of peripheral moiety precursors is selected so that at least about 90% of all possible combinations of n peripheral moieties derived from the subset of peripheral moiety precursors have a molecular mass sum distinct from the molecular mass sums of all of the other combinations of n peripheral moieties. The method further comprises contacting the peripheral moiety precursor subset with a scaffold precursor that has n reactive groups, each of which is capable of reacting with at least one peripheral moiety precursor to form a covalent bond. The peripheral moiety precursor subset is contacted with the scaffold precursor under conditions sufficient for the reaction of each reactive group with a peripheral moiety precursor, resulting in a mass-coded set of compounds of the general formula A(B)ₙ.

Specifically, the method for producing a mass-coded set of compounds of the general formula A(B)ₙ includes the steps of: (a) choosing every set of two different peripheral moiety precursors from a peripheral moiety precursor set, wherein choosing is performed in a manner such that for each set of two, if the two peripheral moiety precursors have equal molecular masses, then one of the two is removed, forming a remaining set; (b) from the remaining set, choosing every set of four peripheral moiety precursors, including for a given set of four, removing one of the four peripheral moiety precursors if the sum of the molecular masses of the first two precursors in the given set of four equals the sum of the molecular masses of the second two precursors in the given set of four peripheral moiety precursors, said choosing forming a remainder set; (c) from the remainder set, choosing every set of six different peripheral moiety precursors, including for a given set of six, removing one of the six peripheral moiety precursors if the sum of the molecular masses of the first three precursors in the given set of six equals the sum of the molecular masses of the second three precursors in the given set of six, said choosing forming a working selection set of peripheral moiety precursors; (d) from the working selection set of peripheral moiety precursors choosing a peripheral moiety precursor subset such that said subset comprises a sufficient number of peripheral moiety precursors that there exist at least about 250 distinct combinations of n peripheral moieties derived from said subset, wherein at least about 90% of the combinations of n peripheral moieties derived from said subset have molecular mass sums which are distinct from the molecular mass sums of all other combinations of n peripheral moieties derived from said subset; and (e) contacting said peripheral moiety precursor subset with a scaffold precursor, said scaffold precursor having n reactive groups, wherein each reactive group is capable of reacting with at least one peripheral moiety precursor to form a covalent bond, under conditions sufficient for the reaction of each reactive group with a peripheral moiety precursor, thereby producing a mass-coded set of compounds of the general formula A(B)ₙ.

In another aspect, a test kinase inhibitor can be identified by screening a mass-coded library (e.g., a pre-existing mass-coded library) produced from a set of individual discrete test kinase inhibitors by retrosynthetically defining the scaffold and building blocks comprised within the set of individual discrete compounds and performing the algorithm as described, supra. (Corey and Cheng, 1995, The Logic of Chemical Synthesis, John Wiley & Sons, Inc.)

In another aspect, a test kinase inhibitor can be identified by screening a library of compounds. The molecular weight for each member of the library can be calculated and the members of the library combined in a mass-coded format, that is, the library of compounds can be prepared such that each member (or essentially all the members, e.g., at least 90% of the members, or at least any integer % between 90-100, inclusive) has a unique mass relative to the other members of the library. The method generally includes contacting members of the mass-coded library with a test kinase (e.g., a kinase in the unactivated state). The bound compounds are then separated from the unbound compounds. In another embodiment, the bound compounds are optionally separated from the unbound compounds. Bound compounds (i.e., potential inhibitors) can then be identified (e.g., by mass-spectrometry). The method can further include determining whether the potential binder inhibits (e.g., renders non-functional or less functional) the test kinase (e.g., in a conventional assay, e.g., biochemical or cell-based assay) including the steps of contacting the inhibitor to the test kinase under conditions and for a time sufficient to allow the inhibitor to bind to the test kinase.

In another aspect, the invention provides a method of designing an inhibitor of a test kinase including the steps of a) using a 3-dimensional structure of the test kinase to locate a DFG motif; b) identifying an allosteric binding site formed by the DFG motif in the DFG-out conformation, wherein the allosteric binding site is spatially distinguishable or distinct from an ATP binding site and an activation loop; and c) designing an inhibitor based on the allosteric binding site. The test kinase can be a kinase whose 3-dimensional structure is known, such as p38 MAP kinase, c-abl, or insulin receptor kinase and which is in the unactivated form. The method can further include determining whether the potential binder inhibits the test kinase including the steps d) contacting the inhibitor to the kinase under conditions and for a time sufficient to allow the inhibitor to bind to the kinase; and e) determining if the kinase is rendered non-functional or less functional by the inhibitor. In another embodiment, the test kinase is not c-abl, p38MAPK, or insulin receptor tyrosine kinase.

In another aspect, the invention provides a method of identifying an inhibitor of a test kinase, including the steps: a) providing a test compound that binds to the test kinase without physically binding (e.g., only partially binds or does not bind at all) to an ATP binding site on the test kinase, wherein the test compound binds to an allosteric site present when a DFG motif of the test kinase is in a DFG-out position; and b) determining if ATP can bind to the ATP binding site on the test kinase, wherein indirectly interfering with the binding of ATP to the test kinase by binding of the test compound to the allosteric site of the test kinase identifies the test compound as a kinase inhibitor; and c) optionally performing a kinase assay in the presence or absence of the inhibitor bound to the test kinase, wherein decreased kinase activity in the presence of the test compound relative to kinase activity in the absence of the test compound further confirms the test compound is an inhibitor of the test kinase. In one embodiment, the test kinase is not c-abl, p38MAPK, or insulin receptor tyrosine kinase.

In another aspect, the invention provides a method of identifying an inhibitor of a test kinase, including the steps: a) providing a test compound that binds to the test kinase without physically binding to an ATP binding site (e.g., only partially binds the ATP binding site or does not bind the ATP binding site) on the test kinase; and b) determining if ATP can bind to the ATP binding site on the test kinase, wherein interfering with the binding of ATP to the test kinase by binding of the test compound to the test kinase identifies an inhibitor of the test kinase, wherein the test compound confines (e.g., locks or holds) a DFG motif of the test kinase in a DFG-out position. In another embodiment, a further step can include confirming the test compound is an inhibitor of a kinase by performing a kinase assay in the presence and absence of the test compound, wherein decreased or absent kinase activity in the presence of the test compound relative to kinase activity in the absence of the test compound confirms identification of a kinase inhibitor. In another embodiment, the test kinase is not c-abl, p38MAPK, or insulin receptor tyrosine kinase.

In another aspect, the invention provides a method of identifying an allosteric binding site spatially distinguishable or distinct from an ATP binding site and an activation loop, the method comprising a) comparing a test kinase to a reference kinase whose 3-dimensional structure is known; b) locating a DFG motif in the test kinase based on its location in the reference kinase (e.g., by computationally overlaying the test kinase and the reference kinase and/or by multiple sequence alignment (Peitsch et al., 1995, ProMod: automated knowledge-based protein modelling tool. PDB Quarterly Newsletter 72:4; Marti-Renom et al., 2000, Annu. Rev. Biophys. Biomol. Struct. 29, 291-325, 2000)); and c) measuring shortest distance between an alpha carbon residue in alpha helix C and a non-backbone heavy atom of phenylalanine, leucine or tryptophan of the DFG motif of the test kinase, wherein a distance of greater than 11 Å and less than 20 Å characterizes the DFG motif of the test kinase in the DFG-out conformation, wherein the DFG motif in the DFG-out conformation identifies an allosteric binding site. When the DFG motif is in the DFG-out position, it can induce the formation of a concave pocket, wherein the surface of the concave pocket is formed in part by a contiguous series of amino acids (designated X through Y) as in amino acids 104 to 111 in c-abl (X being amino acid 104 and Y being amino acid 111 in the case of c-abl). In one embodiment, amino acids X through Y of a test kinase can be homologous to amino acids X through Y of a reference kinase. In another embodiment, amino acids X through Y can consist of a contiguous sequence of amino acids consisting of Leu 104 through Ala111 of a protein whose PDB accession code is 1kv1 (chain A) as determined by sequence alignment analysis. In another embodiment, amino acids X through Y can consist of a contiguous sequence of amino acids consisting of Leu 104 through Ala111 of a protein whose PDB accession code is 1kv2 (chain A) as determined by sequence alignment analysis. In another embodiment, amino acids X through Y of a test kinase consist of amino acids homologous to a contiguous sequence of amino acids consisting of Leu104 through Ala111 of a protein whose PDB accession code is 1kv1 (chain A) or 1kv2 (chain A) as determined by sequence alignment analysis. In another embodiment, amino acids X through Y of the test kinase consist of a contiguous sequence of amino acids consisting of amino acids homologous to Ile313 through Asn322 of 1iep (chain A), 1iep (chain B), 1fpu (chain A), or 1fpu (chain B). In another embodiment, amino acids X through Y of the test kinase consist of a contiguous sequence of amino acids consisting of amino acids homologous to Leu1073 through 1080 of 1irk.

In another aspect, the invention provides a method of identifying an allosteric binding site, spatially distinguishable or distinct from an ATP binding site and an activation loop, for an inhibitor of a test kinase, including the steps: a) locating (e.g., by multiple sequence alignment) a DFG motif in the test kinase by comparing tertiary structure of the test kinase to tertiary structure of a kinase whose 3-dimensional structure is known; b) allowing a test compound to bind to the allosteric site when the DFG motif is in a DFG-out position; c) determining if the test compound inhibits the activity of the test kinase (e.g., by kinase assay or producing and analyzing an x-ray crystal of the test compound bound to the kinase); and d) determining if the test compound binds the test kinase at an allosteric site formed when the DFG motif is in a DFG-out conformation (e.g., by labeling the test compound), wherein binding of the test compound such that the DFG motif is confined in the DFG-out position, identifies the allosteric binding site of the test kinase. In another embodiment, the test kinase is not c-abl, p38MAPK, or insulin receptor tyrosine kinase.

In another aspect, the invention provides a method of identifying an inhibitor by a) comparing a test kinase (e.g., those of Table 2) to a kinase whose 3-dimensional structure is known; b) identifying an allosteric binding site on the test kinase; c) designing a scaffold targeting the allosteric binding site; d) providing mixtures of compounds based on the scaffold; and e) identifying ligands for the allosteric site by affinity screens against the kinase. This method can further include the step of f) demonstrating kinase inhibitory activity of the inhibitor. In another embodiment, the test kinase is not c-abl, p38MAPK, or insulin receptor tyrosine kinase. In another embodiment the kinase whose 3-dimensional structure is known is selected from the group consisting of kinases with Protein Data Bank identifiers 1iep chain a, 1iep chain b, 1fpu chain a, 1fpu chain b, lkyl chain a, 1kv2 chain, 1irk, 1gn3n chain a, and 1g3n chain b. In another embodiment, the allosteric binding site is spatially distinct from the ATP binding site and the activation loop. In yet another embodiment, the allosteric binding site is identified by locating the DFG motif (e.g., DFG, DLG, or DWG) and the DFG motif is in the DFG-out conformation.

In another aspect, the invention provides a method of identifying an allosteric binding site, spatially distinguishable from an ATP binding site and an activation loop, for a test kinase inhibitor, the method comprising: a) allowing a test compound to bind to the test kinase whose DFG motif is in the out position; b) determining if the test compound inhibits the activity of the test kinase; and c) determining if a known inhibitor of the test kinase, the known inhibitor binding an allosteric site formed by the DFG motif in the DFG-out position. The known inhibitor can compete with the test compound for test kinase binding, wherein competitive binding by the known inhibitor identifies the allosteric binding site, spatially distinguishable or distinct from an ATP binding site and an activation loop, for a test kinase inhibitor. In one embodiment, the test kinase is not c-abl, p38MAPK, or insulin receptor tyrosine kinase.

In another aspect, the invention provides a method of synthesizing a kinase inhibitor, the method comprising: a) comparing a test kinase to a kinase whose 3-dimensional structure is known; b) identifying an allosteric binding site; c) designing an inhibitor based on the allosteric binding site; and d) synthesizing the inhibitor. In one embodiment, the test kinase is not c-abl, p38MAPK, or insulin receptor tyrosine kinase.

In another aspect the invention provides an inhibitor designed by the method of a) comparing a test kinase to a kinase whose 3-dimensional structure is known (e.g., Protein Data Bank identifiers: lkyl chain a, 1kv2 chain a, 1iep chain a, 1iep chain b, 1fpu chain a, 1fpu chain b, and 1irk); b) identifying an allosteric binding site; and c) designing an inhibitor based on the allosteric binding site. In another embodiment, the test kinase is not c-abl, p38MAPK, or insulin receptor tyrosine kinase.

In another aspect the invention provides a pharmaceutical composition comprising the inhibitor designed by the method of a) comparing a test kinase to a kinase (e.g., a reference kinase) whose 3-dimensional structure is known (e.g., Protein Data Bank identifiers: 1kv1 (chain A), 1kv2 (chain A), 1iep (chain A), 1iep (chain B), 1fpu (chain A), 1fpu (chain B), and 1irk); b) identifying an allosteric binding site (e.g., a concave pocket identified by test kinase amino acids analogous to amino acids X through Y of a reference kinase (see Table 1)); and c) designing an inhibitor based on the allosteric binding site (e.g., the concave pocket). In another embodiment the pharmaceutical composition can comprise an inhibitor designed by the method of a) using a 3-dimensional structure of a reference kinase to locate a DFG motif in the test kinase; b) identifying an allosteric binding site formed by the DFG motif in the DFG-out conformation, wherein the allosteric binding site is spatially distinguishable from an ATP binding site and an activation loop; and c) designing an inhibitor based on the allosteric binding site. In another embodiment, the test kinase is not c-abl, p38MAPK, or insulin receptor tyrosine kinase.

In another aspect, the invention provides for a kit comprising the inhibitor designed, discovered, or identified by the any of the methods described herein. For example, the method of a) comparing a test kinase to a kinase whose 3-dimensional structure is known (e.g., Protein Data Bank identifiers: 1kv1 (chain A), 1kv2 (chain A), 1iep (chain A), 1iep (chain B), 1fpu (chain A), 1fpu (chain B), and 1irk); b) identifying an allosteric binding site; and c) designing an inhibitor based on the allosteric binding site. In another embodiment the kit can comprise an inhibitor designed by the method of a) using a 3-dimensional structure of the test kinase to locate a DFG motif; b) identifying an allosteric binding site formed by the DFG motif in the DFG-out conformation, wherein the allosteric binding site (e.g., a concave pocket induced by DFG in the DFG-out position) is spatially distinguishable from an ATP binding site and an activation loop; and c) designing an inhibitor (e.g., computationally) based on the allosteric binding site (e.g., a concave pocket induced by DFG in the DFG-out position).

In another aspect, the invention provides a method of inhibiting kinase activity or treating a kinase-mediated disease or disease symptoms in a subject (e.g., a mammal, e.g., a human), comprising the step of administering to the subject a compound comprising an inhibitor designed by the method of a) comparing a test kinase to a kinase whose 3-dimensional structure is known (e.g., Protein Data Bank identifiers: 1kv1 (chain A), 1kv2 (chain A), 1iep (chain A), 1iep (chain B), 1fpu (chain A), 1fpu (chain B), and 1irk); b) identifying an allosteric binding site (e.g., a concave pocket induced by DFG in the DFG-out position); and c) designing an inhibitor based on the allosteric binding site (e.g., a concave pocket induced by DFG in the DFG-out position). In another embodiment the method of inhibiting kinase activity in a subject (e.g., a mammal, e.g., a human) can comprise an inhibitor designed by the method of a) using a 3-dimensional structure of the test kinase to locate a DFG motif; b) identifying an allosteric binding site (e.g., a concave pocket induced by DFG in the DFG-out position) formed by the DFG motif in the DFG-out conformation, wherein the allosteric binding site (e.g., a concave pocket induced by DFG in the DFG-out position) is spatially distinguishable from an ATP binding site and an activation loop; and c) designing an inhibitor based on the allosteric binding site (e.g., computationally).

In another aspect, the invention provides a method of making a pharmaceutically useful composition comprising combining an inhibitor (e.g., an inhibitor identified by any of the methods described herein) with one or more pharmaceutically acceptable carriers and optionally further comprising combining an additional therapeutic agent. The pharmaceutically useful composition can contain more than one kinase inhibitor and/or more than one additional therapeutic agent. In one embodiment, the inhibitors identified can be used for the preparation of a medicament for use in treating disease (e.g., disease classes such as cancer (e.g., breast cancer, prostate cancer, lung cancer), inflammation (e.g., arthritis, rheumatoid arthritis), neurological disorders (e.g., Alzheimer's disease), and obesity).

Definitions

The “unactivated form” or “basal form” of a biomolecule such as an enzyme (i.e., target moiety, e.g., kinase) is the state of the target moiety in which it is in a conformation unable or less able to perform its physiological function. Typically, the unactivated or basal form of a biomolecule needs a further step of transformation to gain full biological activity. A further step can include, for example in a kinase or other biomolecule for which it is appropriate, phosphorylation, dephosphorylation, other modification, binding to another monomer, multimerization, localization, translocation, binding to a cofactor, or binding to the substrate, among other events that can activate an unactivated biomolecule. As such, an unactivated biomolecule is competent to be activated by any one of these events or other events. If the target moiety is an enzyme, the unactivated form or basal form is generally the state of the enzyme that is unable to perform its function on its substrate (e.g., a kinase in a state unable or less able to phosphorylate its substrate; a methylase in a state that is unable or less able to methylate its substrate; a phosphatase in a state unable or less able to dephosphorylate its substrate; a protease unable or less able to perform its proteolytic function; a polypeptide that is not associated with another polypeptide or molecule; a polypeptide is not selectively mutated or induced into a specific conformation (e.g., induced by temperature change or other factor)). In some cases, the unactivated form or basal form is not bound to ATP. In other cases the unactivated or basal form is in a monomeric state rather than a multimeric state or vice versa. In still other cases, the unactivated or basal form is or is not bound to a cofactor or other molecule essential in allowing it to perform its function or the unactivated or basal form is not present in an induced conformation. A target moiety in the unactivated or basal state may still have activity or function but its activity or function relative to that of its active state is less or decreased, whether in magnitude and/or duration of activity or function. As used herein, “unactivated form” refers to either the unactivated form or the basal form of the target moiety. Though the two terms differ in their meaning, they are both used equivalently within the context of the described methods and compositions. Namely, the described methods can use a target moiety which is in the unactivated form or it can use a target moiety in the basal form and in either case, the same result can be achieved; that is, the identification of a compound that binds to the target moiety in that particular state.

On the other hand, a biomolecule can be “inactive” and this refers to the biomolecule lacking the capacity to be activated. Such events that can cause a biomolecule to be inactive include mutation, truncation, or other modification which causes the biomolecule to be unable to be activated under any set of conditions or in the presence of any activating species.

An “unactivated kinase” is a kinase with a lesser ability to bind ATP and/or to perform its functional activity including its in vivo functional activity. As a result, an unactivated kinase has less kinase activity or no kinase activity relative to when it performs its in vivo function. An unactivated kinase can be a kinase which requires any one or a number of the following events: modification (e.g., phosphorylation, dephosphorylation, other modification), contact with or binding to a regulating moiety (e.g., a cofactor, a protein/lipid regulator) or other event (e.g., multimerization, localization, translocation) in order to become fully activated. As such, an unactivated kinase is fully competent to become fully functional or activated upon the completion of the aforementioned event or events. However, an inactive kinase is one which is rendered incompetent in becoming activated, whether by mutation, truncation, misfolding, inability to localize appropriately, or inability to bind a required cofactor or other required molecule for activation. In any case, the methods described herein can be used, for example, for the identification of a kinase inhibitor which binds a novel allosteric site on the kinase which may be present when the kinase is unactivated or inactive.

The term “calibrating” refers to the determination, by measurement or comparison with a standard, of the correct value of each scale reading on a meter or other measuring instrument. For example, an analytical or measuring instrument (e.g., a mass spectrometer) can be calibrated by measuring or determining a plurality of values of an analyte (e.g., concentrations of ligands) whose true values are known.

The term “mass spectrometer” refers to an analytical device that uses the difference in mass-to-charge ratio (m/e) of ionized atoms or molecules to separate them from each other. Mass spectrometry is therefore useful for quantitation of atoms or molecules and also for determining chemical and structural information about molecules. Molecules have distinctive fragmentation patterns that provide structural information to identify structural components. The general operation of a mass spectrometer is: (a) create gas-phase ions; (b) separate the ions in space or time based on their mass-to-charge ratio, and (c) measure the quantity of ions of each mass-to-charge ratio. The ion separation power of a mass spectrometer is described by its resolution.

There are many ionization sources known in the art, for example, electrospray ionization (ESI), electron ionization (E1), fast atom bombardment ionization (FAB), matrix-assisted laser desorption (MALDI), electron-capture (sometimes called negative ion chemical ionization or NICI), and atmospheric pressure chemical ionization (ApCI). The ions produced in any of the ionization methods above are passed through a mass separator, typically a magnetic field, a quadrupole electromagnet, or a time-of-flight mass separator so that the mass of the ions may be distinguished as well as the number of ions at each mass level.

Mass spectrometry (MS) is a widely used technique for the characterization and identification of molecules, both in organic and inorganic chemistry. MS provides molecular weight information about a molecule. The molecular weight of a molecule is a crucial piece of information in the identification of a particular molecule in a mixture of molecules. MS analysis can be used, for example, in drug development and manufacture, pollution control analysis, and chemical quality control.

The term “ligand” refers to a molecule that associates or binds with a receptor (e.g., interacts in a covalent or non-covalent manner). In some cases, the binding of the ligand to the receptor can have a biological effect (e.g., agonism or antagonism). For example, the ligand can be a polypeptide (e.g., a protein) binding to a biomolecule (e.g. DNA molecule) wherein the binding of the protein to the DNA has initiates mRNA synthesis. The ligand can also be an organic molecule (e.g., a pharmaceutical compound) bound to an enzyme (e.g., HIV protease) wherein the binding of the organic molecule to the enzyme inhibits enzymatic activity.

When a ligand is a “binder” or “binds” a target or is a ligand of a target, the association can be by covalent interaction, non-covalent interaction, or other interaction, including a variety of forms of interaction (e.g., steric interaction, van der Waals interaction, electrostatic interaction, solvation interaction, charge interaction, covalent bonding interaction, non-covalent bonding interaction (e.g., hydrogen-bonding interaction), entropically or enthalpically favorable interaction).

An “inhibitor” is a molecule (e.g., a small molecule, e.g., less than about 5 kDa in size) that, when it binds to a target (e.g., a kinase), can decrease physiological activity of the target (e.g., render a kinase less functional) or block its activity (e.g., render a kinase non-functional). An inhibitor can be a small molecule of less than 1000 daltons, a small molecule less than 750, 600 or 500 daltons, a polypeptide of naturally occurring or not naturally occurring amino acids, a peptide of naturally occurring or not naturally occurring amino acids, a peptoid, a peptidomimetic, a synthetic compound, a synthetic organic compound, or the like. For example, a target (e.g., an enzyme, e.g., a kinase) which is rendered “less functional” by an inhibitor refers to a target (e.g., an enzyme, e.g., a kinase) having detectable activity which is less than its activity under physiological conditions. A target (e.g., an enzyme, e.g., a kinase) is rendered “non-functional” by an inhibitor if its activity is not detectable by a biological assay (e.g., an enzyme inhibition assay, e.g., a kinase inhibition assay).

An “allosteric site” on a target, for example a kinase, as described herein, is a site that is spatially distinct from the ATP binding site of the target (e.g., the kinase) that when occupied by a ligand (e.g., allosteric ligand) modulates (e.g., inhibits) or prevents ATP binding and, thus, kinase function. A spatially distinct “allosteric site” on a target (e.g., a kinase) can also modulate or prevent substrate binding with similar effects on target (e.g., kinase) function. “Spatially distinct from the ATP binding site” refers to a binding site that is separate from the ATP binding site and is defined by amino acid residues within the kinase that taken together are not identical to the sequence of amino acids that define the ATP binding site. A site that is spatially distinct from the ATP binding site can differ from the ATP binding site in length of amino acids, can differ by a single amino acid, and can differ in the order of amino acids in the sequence comprising the site. The allosteric site is thus spatially distinct or distinguishable from the ATP binding site (e.g., the ATP binding site and the allosteric binding site are not one in the same). It is also possible that the allosteric site of the invention partially overlaps the ATP site but the allosteric site and the ATP binding site are not to be construed as one in the same. The methods described herein can be applied to identifying ligands (e.g., inhibitors) that bind to a target (e.g., a kinase) at an allosteric site and by binding the allosteric site, these ligands (e.g., inhibitors) lock, confine, or hold the DFG motif in the DFG-out position. The DFG in the DFG-out position physically prevents ATP from binding to the target (e.g., kinase), thus allowing for inhibition of the target (e.g., kinase) in this novel manner.

The methods described herein can be applied to the identification of inhibitors of enzymes (e.g., kinases). “Designing an inhibitor based on the allosteric binding site” refers to the use of computer modeling to determine the optimal characteristics of a potential enzyme (e.g., kinase) binder based on the allosteric binding site (e.g., a concave pocket induced by DFG in the DFG-out position) and using that information to synthesize the potential enzyme (e.g., kinase) binder. The potential enzyme (e.g., kinase) binder is designed to bind to the allosteric binding site (e.g., a concave pocket induced by DFG in the DFG-out position) which is present when the DFG motif is in the DFG-out conformation. Designing an inhibitor can also mean the design of a scaffold with the correct topology (shape) and electronic properties to fit the allosteric binding site. Once the scaffold is designed, a mass-coded library (e.g., one based on that scaffold) can be provided or synthesized and allosteric binders (e.g., inhibitors) from this library are identified by affinity screening with the kinase under conditions where the allosteric site (i.e., present when the DFG is in the DFG-out position) predominates (i.e., unactivated kinase in the absence of ATP and substrate). It was a surprising result, and a key aspect of the present invention, that under conditions in which the kinase is in the unactivated form, affinity screening methods provided inhibitors that preferentially target kinases in the DFG-out conformation.

The term “organic molecule” refers to a compound wherein the molecule includes carbon and hydrogen, and can also include additional elements such as nitrogen, oxygen, phosphorus, halogens, or sulfur (e.g., an pharmaceutical compound). Pharmaceutically acceptable salts (e.g., maleic, hydrochloric, hydrobromic, phosphoric, acetic, fumaric, salicylic, citric, lactic, mandelic, tartaric and methanesulfonic) are also encompassed within the meaning of the term “organic molecule.”

The term “receptor” refers to a biomolecule to which a ligand can bind and exert a signaling function within a cell. The ligand can stimulate or activate a normal physiologic function. Alternatively, the ligand can modulate or inhibit a physiologic function. The receptor, upon association with a second molecule, enables or initiates an effect (e.g., biological activity or detectable signal). For example, a receptor can be a protein that binds a specific extracellular signal molecule (e.g., a ligand) and initiates a response in the cell. Examples of cell-surface receptors include the acetylcholine receptor and the insulin receptor. Examples of intracellular receptors include hormones, which can bind ligands that diffuse into the cell across the plasma membrane. Other examples of receptors include: polypeptides, proteins, enzymes, ribozymes, RNA, DNA, and biomolecular mimics.

The term “biomolecule” refers to a molecule having an effect on biological activity (e.g., metabolism, antagonism, agonism, signaling, or transcription). While a biomolecule can be found in the body, the term biomolecule is not limited to naturally occurring biomolecules, but rather includes synthetic versions of naturally occurring biomolecules as well as fragments and modifications thereof. Examples of biomolecules include: polypeptides, proteins, enzymes, ribozymes, RNA, and DNA.

The term “polypeptide” refers to a polymer composed of multiple amino acids. A protein can be an example of a polypeptide.

The term “enzyme” refers to a macromolecule, usually a protein, that functions as a (bio) catalyst by increasing the reaction rate. In general, an enzyme catalyzes only one reaction type (i.e., reaction selectivity) and operates on only one type of substrate (i.e., substrate selectivity). Substrate molecules are transformed at the same site (regioselectivity) and generally; only one chiral substrate of a racemic substrate pair is transformed (enantioselectivity, a special form of stereoselectivity).

The term “nucleic acid” refers to a polymer composed of nucleotide subunits. The nucleotide subunits can be joined together through phosphodiester bonds.

The term “receptor-ligand pair” refers to a complex consisting of a receptor and a ligand that are generally held together in a reversible manner, by noncovalent interactions (e.g., hydrogen bonding, ionic interactions, or hydrophobic interactions).

The term “equilibrium” refers to a state in a reversible chemical and/or biochemical reaction and/or interaction at which the reactants are turning into products at the same rate as the products are turning back into the reactants, so that the amounts of each reactant and product remains essentially constant. Sampling a mixture over a period of time and determining that the ratio of starting material to products has not changed can rigorously prove that the reaction has reached equilibrium.

The term “size-exclusion-chromatography” (SEC) refers to the use of porous particles to separate molecules of different sizes. It is generally used to separate biological molecules, and to determine molecular weights and molecular weight distributions of polymers. Generally, molecules that are smaller than the pore size can enter the particles and therefore have a longer path and longer transit time than larger molecules that cannot enter the particles. Molecules larger than the pore size cannot enter the pores and elute together as the first peak in the chromatogram. This condition is called total exclusion. Molecules that can enter the pores will have an average residence time in the particles that depends on the molecule's size and shape. Different molecules therefore have different total transit times through the column. Molecules that are smaller than the pore size can enter all pores, and have the longest residence time on the column and elute together as the last peak in the chromatogram.

The term “liquid chromatography” refers to an analytical chromatographic technique that is used to separate ions and/or molecules that are dissolved in a solvent. If the sample solution is in contact with a second solid or liquid phase, the different solutes will interact with the other phase to differing degrees due to differences in adsorption, ion exchange, partitioning, or size. These differences allow the mixture components to be separated from each other by using these differences to determine the transit time of the solutes through a column. High-performance liquid chromatography (HPLC) is a form of liquid chromatography to separate compounds that are dissolved in solution. HPLC instruments consist of a reservoir of mobile phase, a pump, an injector, a separation column, and a detector. Injecting a plug of the sample mixture onto the column separates compounds. The different components in the mixture pass through the column at different rates due to differences in their partitioning behavior between the mobile liquid phase and the stationary phase.

The term “competitive binder” refers to a ligand that binds to a receptor at a specific site (e.g., a catalytic site of an enzyme), where it competes with another ligand for binding in a dynamic, equilibrium-like process.

The term “target moiety” refers to a biomolecule or its fragment against which a screening process can be applied to discover binding ligands.

The term “target form” refers to a distinctive biochemical or biophysical state of a biomolecule (e.g., a kinase) such as different conformations, aggregation states, chemical modifications, associations with other biomolecules or cofactors, and associations with one or more ligands.

The target herein can refer to a kinase. “DFG motif” refers to a conserved motif in kinases that consists of three contiguous amino acids, aspartate followed by phenylalanine, tryptophan or leucine, followed by glycine (i.e., Asp-Phe/Leu/Trp-Gly). Thus the DFG motif can have the amino acid sequence DFG, DWG or DLG. For example, Asp1150-Phe1151-Gly1152 is the DFG motif in insulin receptor kinase; Asp381-Phe382-Gly383 is the DFG motif in c-abl; and Asp168-Phe169-Gly170 is the DFG motif in p38MAPK. This motif can be identified by a multiple sequence alignment of proteins whose DFG motif location is known with those whose DFG motif are to be determined.

A unique structure in a specific target, a kinase is the “helix alpha-C” (also referred to as helix α-C). “Helix α-C” refers to a conserved helix in kinases. For example, in insulin receptor kinase, this helix consists of the amino acid sequence, LRERIEFLNEASVM (amino acids 1038 to 1051) with the Val-Met motif at positions 1050-1051. In another example, in c-abl, this helix consists of the amino acid sequence, VEEFLKEAAVM (amino acids 280 to 290) with the Val-Met motif located at amino acids 289-290. The helix α-C of a test kinase is “homologous to” the helix α-C in insulin receptor kinase which contains valine at position 1050 followed by methionine at position 1051 and is also “homologous to” the helix α-C in c-abl which contains valine at position 289 and methionine at position 290. 1irk is the accession code for insulin receptor kinase in the Protein Data Bank (PDB) which can be found on the world wide web at the address rcsb.org/pdb/ and 1iep can also be found at this address and is the PDB accession code for c-abl bound to STI-571 (i.e., Gleevec®).

Families of amino acid residues having similar side chains have been defined in the art. These similarities may allow the amino acids to act “analogously” within the three-dimensional structure of the kinase. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, for example, analogous amino acids to lysine would be arginine or histidine. As another example, aspartic acid is analogous to glutamic acid as described herein.

The details of one or more embodiments of the invention are set forth in the description below. Other features, objects, and advantages of the invention will be apparent from the description and from the claims.

### DETAILED DESCRIPTION

The methods described herein are based on the finding that the forms of a target (e.g., biomolecule) other than the activated form can be very useful in the identification of novel inhibitors of the target (e.g., biomolecule) function. The methods provide for screening targets in a novel fashion to discover compounds that bind to a low activity or inactive state of a target and act as inhibitors of the target function in subsequent biological assays. The process can be applied to all types of biological targets including nucleic acid (e.g., DNA or RNA) or proteins (e.g., membrane-associated proteins, enzymes, nuclear hormone receptors (NHRs), and G-protein coupled receptors (GPCRs)). The methods described do not necessitate a biochemical assay for its output and utilize only very small quantities of a purified target. The methods described herein confer several advantages, including: no prior knowledge the target's structure is necessary; mixtures of compounds can be used; and, multiple forms of a given target can be multiplexed together (e.g. combining and screening two or more distinct forms of a protein in one common binding reaction) to expedite the efficiency of the process. The methods described herein can utilize mass-coded combinatorial libraries and Automated Ligand Identification System (ALIS), described infra.

U.S. Pat. No. 6,207,861 relates to methods for making mass-coded combinatorial libraries and methods of identifying (i.e., screening) compounds in those mass-coded combinatorial libraries that associate with one or more biomolecules. The mass-coded libraries can be designed and synthesized such that at least about 90% of the individual compounds have a molecular mass sum that is distinct from the molecular mass sums of the other individual compounds in the mass-coded library, and in this case redundancy is tolerated (e.g., mass-coding sorts for mass unique scaffold+peripheral moiety combinations, and thus there is mass redundancy between positional isomers that is counted). In other instances, the mass-coded library can be designed such that at least 90% of the individual compounds have a molecular sum that is distinct from the molecular mass sums of the other individual compounds of the mass-coded library where redundancy is not tolerated (e.g., mass-coding sorts for mass unique scaffold+peripheral moiety combinations, and thus there is mass redundancy between positional isomers that is not counted).

The screening methods in U.S. Pat. No. 6,207,861 describe a system referred to as the Automated Ligand Identification System (ALIS). The ALIS system generally functions as follows: (1) a dilute solution of the biomolecule of interest (e.g. a protein) is incubated in the presence of a ligand (e.g. a small organic molecule or library thereof) for a prescribed length of time to allow the biomolecule-ligand complex forming reaction to reach equilibrium; (2) the solution of biomolecule, unbound ligand and biomolecule-ligand complex is passed through a size exclusion chromatography stage to separate the biomolecule plus biomolecule-ligand complex from unbound ligand on the basis of molecular size, with the biomolecule plus biomolecule-ligand complex co-eluting at the front of the eluant stream; (3) the portion of the eluant stream containing the biomolecule plus biomolecule-ligand complex but not containing any unbound ligand is diverted to a reverse-phase chromatography stage for desalting and elution into a mass spectrometer where the small molecule may be identified on the basis of its molecular weight or mass spectrometry-mass spectrometry fragmentation pattern and quantified by measurement of its signal response.

As described, supra, in identifying target ligands (e.g., inhibitors of kinases), mixtures of compounds, scaffolds, or libraries are incubated with the test target, then target-bound compounds are separated from unbound compounds before identification by mass-spectrometry. In one embodiment, the target-bound compounds are then separated in order to identify the bound component. Disruption of the target-compound binding pairs can be accomplished by a variety of methods such as use of chromatography (e.g., high resolution reverse phase chromatography under high temperature) change of pressure, pH, salt concentration, temperature or organic solvent concentration; or competition with a known target binding agent, or any combination of these techniques.

Once the target-bound compounds are separated from the target, the compounds can be identified by mass-spectrometry. U.S. Pat. No. 6,147,344 describes methods for analyzing mass spectrometer data in which a control sample measurement is performed providing a background noise check. The peak height and width values at each m/z ratio as a function of time are stored in a memory. A mass spectrometer operation on a material to be analyzed is performed and the peak height and width values at each m/z ratio versus time are stored in a second memory location. The mass spectrometer operation on the material to be analyzed is repeated a fixed number of times and the stored control sample values at each m/z ratio level at each time increment are subtracted from each corresponding one from the operational runs, thus producing a difference value at each mass ratio for each of the multiple runs at each time increment. If the MS value minus the background noise does not exceed a preset value, the m/z ratio data point is not recorded, thus eliminating background noise, chemical noise and false positive peaks from the mass spectrometer data. The stored data for each of the multiple runs is then compared to a predetermined value at each m/z ratio and the resultant series of peaks, which are now determined to be above the background, is stored in the m/z points in which the peaks are of significance.

Specifically, U.S. Pat. No. 6,147,344 describes a technique for automatically analyzing mass spectrographic data from mixtures of chemical compounds consisting of a series of screens designed to eliminate or reduce incorrect peak identifications due to background noise, system resolution, system contamination, multiply charged ions and isotope substitutions. This method allows for the identification of organic compounds in complex mixtures of organic compounds and so is useful for the methods described herein. The technique performs a mass spectrum operation on a control sample, producing a first group of output values. Next, a mass spectrographic operation on a sample to be analyzed, is performed, producing a second group of output values. Select a first m/z ratio for a material expected to be present in the mixture from a predetermined library of calculated mass spectrometer output spectrums and subtract the value of the control sample at the expected output value from the value of the analyzed sample, and compare the difference to a predetermined value. If the value is greater than the predetermined value thus indicating that the signal is above the background noise level, generating a record at that m/z value for an expected material. The same mass spectrum operation is performed several times to eliminate random noise and background contamination. Next, peak values that don't have the expected peak width or proper retention time for the separation method are identified. Multiply charged ions are identified by examining peak separation. The m/z location of the expected material is examined and intensity at the expected m/z location is compared with the intensity at the next lower m/z recorded peak to identify peaks related to atomic isotope substitution. With such a technique, mass spectrograph data analysis may be greatly simplified by the identification of probable spurious signals, and analysis will become simpler and more accurate.

Computer Modeling

Upon determination of the three-dimensional structure of a crystal of a reference target-ligand complex, or a reference target, a potential inhibitor can be evaluated by any of several methods, alone or in combination. Such evaluation can utilize visual inspection of a three-dimensional representation of the relevant site, based on the coordinates of the structure described herein, on a computer screen. Evaluation, or modeling, can be accomplished through the use of computer modeling techniques, hardware, and software known to those of ordinary skill in the art. This can additionally involve model building, model docking, or other analysis of target-ligand interactions using software including, for example, QSC, FlexX (Lengauer, Rarey, 1996) or Autodock (Morris et.al., 1998), GLIDE, Modeler, or Sybyl, followed by energy minimization and molecular dynamics with standard molecular mechanics forcefields including, for example, CHARMM and AMBER. The three-dimensional structural information of a target-ligand complex can also be utilized in conjunction with computer modeling to generate computer models of other target protein structures, particularly those with homology to the target from which the three-dimensional structural information was determined. Computer models of target protein structures can be created using standard methods and techniques known to those of ordinary skill in the art, including software packages described herein.

Once the three-dimensional structure of a crystal comprising a protein-ligand complex formed between a target and a standard ligand for that target is determined, a potential ligand is examined through the use of computer modeling using a docking program such as QSC, FlexX, or Autodock to identify potential ligands and/or inhibitors to the allosteric binding site to ascertain how well the shape and the chemical structure of the potential ligand will interact with the binding site. Computer programs can also be employed to estimate the attraction, repulsion, and steric hindrance of the two binding partners (i.e., the allosteric-binding site and the potential ligand). Generally complementary fit, lower steric hindrances, and greater attractive force between the potential ligand and the allosteric binding site are consistent with a tighter binding constant between the two. Furthermore, the more specificity in the design of a potential drug, the more likely that the drug will not interact as well with other proteins. This will minimize potential side-effects due to unwanted interactions with other proteins.

A variety of methods are available to one skilled in the art for evaluating and virtually screening molecules or chemical fragments appropriate for associating with a protein, particularly, for example, a kinase, a phosphatase, a transferase, a GPCR, an NHR, and the like. Such association can be in a variety of forms including, for example, steric interactions, van der Waals interactions, electrostatic interactions, solvation interactions, charge interactions, covalent bonding interactions, non-covalent bonding interactions (e.g., hydrogen-bonding interactions), entropically or enthalpically favorable interactions, and the like.

Numerous computer programs are available and suitable for rational drug design and the processes of computer modeling, model building, and computationally identifying, selecting and evaluating potential inhibitors in the methods described herein. These include, for example, QSC (WO 01/98457), FlexX, Autodock, Glide, Accelrys' Discovery Studio, or Sybyl. Potential inhibitors can also be computationally designed “de novo” using such software packages as QSC (WO 01/98457), Accelrys' Discovery Studio, Sybyl, ISIS, ChemDraw, or Daylight. Compound deformation energy and electrostatic repulsion, can be evaluated using programs such as GAUSSIAN 92, AMBER, QUANTA/CHARMM, AND INSIGHT II/DISCOVER.

These computer evaluation and modeling techniques can be performed on any suitable hardware including for example, workstations available from Silicon Graphics, Sun Microsystems, and the like. These techniques, methods, hardware and software packages are representative and are not intended to be comprehensive listing.

Other modeling techniques known in the art can also be employed in accordance with this invention. See for example, QSC (WO 01/98457), FlexX, Autodock, Glide, Accelrys' Discovery Studio, or Sybyl and software identified at various internet sites (e.g.,
netsci.org/Resources/Software/Modeling/CADD/
c.cam.ac.uk/SGTL/software.html
cmm.info.nih.gov/modeling/universal_software.html
dasher.wustl.edu/tinker/
zeus.polsl.gliwice.pl/˜nikodem//linux4chemistry.html
nyu.edu/pages/mathmol/software.html
msi.umn.edu/user_support/software/MolecularModeling.html
us.expasy.org/
sisweb.com/software/model.htm).

A potential inhibitor is selected by performing rational drug design with the three-dimensional structure (or structures) determined for the allosteric site of a target described herein, in conjunction with or solely by computer modeling and methods described above. The potential inhibitor is then obtained from commercial sources or is synthesized from readily available starting materials using standard synthetic techniques and methodologies known to those of ordinary skill in the art. The potential inhibitor is then assayed to determine its ability to inhibit the target enzyme (e.g., kinase) and/or enzyme pathway (e.g., kinase pathway) as described above.

A potential inhibitor can also be selected by screening a library of compounds (e.g., a combinatorial library, e.g., a mass-coded combinatorial library) as described above. The library of compounds can be screened by affinity screening in which members with the slowest dissociation rates and greatest affinity to a particular protein at the new allosteric site can be selected. ALIS, also described above, can be used to screen the library of compounds. Because the allosteric site is present in many targets when the target is in the unactivated state, ALIS is particularly advantageous. ALIS can work with mixtures of compounds and can specifically allow the identification of bound ligands when the target is in the unactivated state. Conventional target screens often rely on the target of interest being in the activated state.

Pharmaceutical Compositions

Pharmaceutical compositions of this invention comprise a compound identified by a method or methods described herein or a pharmaceutically acceptable salt thereof;
optionally an additional agent selected from a target inhibitory agent (small molecule, polypeptide, antibody, etc.), an immunosuppressant, an anti-cancer agent, antiinflammatory agent, or an anti-vascular hyperproliferation compound, a compound to treat neurological disorders, and an anti-obesity compound; and any pharmaceutically acceptable carrier, adjuvant or vehicle. Alternate compositions of this invention comprise a compound identified by a method or methods described herein or a pharmaceutically acceptable salt thereof; and a pharmaceutically acceptable carrier, adjuvant or vehicle.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tween or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-.beta.-cyclodextrins, or other solubilized derivatives may also be used advantageously to enhance delivery of compounds identified by a method or methods described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions of this invention may be orally administered in any orally acceptable dosage form including, but not limited to, capsules, tablets, emulsions and aqueous suspensions, dispersions and solutions. In the case of tablets for oral use, commonly used carriers include lactose and corn starch. Lubricating agents, such as magnesium stearate, are also typically added. For oral administration in a capsule form, useful diluents include lactose and dried cornstarch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may comprise formulations utilizing liposome or microencapsulation techniques. Such techniques are known in the art.

The pharmaceutical compositions of this invention may also be administered in the form of suppositories for rectal administration. These compositions can be prepared by mixing a compound of this invention with a suitable non-irritating excipient that is solid at room temperature but liquid at rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene-polyoxypropylene compound, emulsifying wax, and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Dosage levels of between about 0.01 and about 100 mg/kg body weight per day, alternatively between about 0.5 and about 75 mg/kg body weight per day of the target inhibitory compounds described herein are useful in a monotherapy and/or in combination therapy for the prevention and treatment of target mediated disease. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

As the skilled artisan will appreciate, lower or higher doses than those recited above may be required. Specific dosage and treatment regimens for any particular patient will depend upon a variety of factors, including the activity of the specific compound employed, the age, body weight, general health status, gender, diet, time of administration, rate of excretion, drug combination, the severity and course of the disease, condition or symptoms, the patient's disposition to the disease, condition or symptoms, and the judgment of the treating physician.

### EXAMPLES

### Example 1

### Screening of a Target Enzyme in the Monomeric form to Identify Ligands that Inhibit Multimeric Enzymatic Activity

The human enzyme inducible nitric oxide synthase (iNOS) is a known to play an important role in inflammation, and thus inhibitors of iNOS activity are candidates for anti-inflammatory drug discovery. For full activity, the iNOS enzyme must exist as a cofactor-loaded homodimer that provides two functional active sites per multimer. In the absence of one required cofactor, tetrahydrobiopterin (THB), the enzyme is known to exist in a low activity form that equilibrates between inactive monomer and the active dimer (Ghosh et al, Biochemistry, 35:1444-9 (1996)). Thus, function-based screening has employed the THB-bound, high activity dimer for inhibitor discovery. However, affinity-based screening of the THB-free, low activity form of iNOS yielded novel ligands that bind to the monomeric form of iNOS and impair dimerization. These ligands act as functional inhibitors of iNOS in both biochemical and cellular assays, with IC50s ranging from 30 nM to 30 uM in a radioligand displacement assay (with a known monomer-specific ligand), and EC50s ranging from 100 nM-50 uM in a LPS-stimulated RAW cell assay.

### Example 2

### Screening of a Target Enzyme in the Absence of a Required Cofactor to Identify Ligands that Inhibit Enzymatic Activity in a Cofactor-Independent Manner

The human enzyme inosine monophosphate dehydrogenase (IMPDH) is known to play an important role in inflammation, and thus inhibitors of IMPDH activity are candidates for anti-inflammatory drug discovery. For full activity, the IMPDH enzyme utilizes the cofactor NAD+ to oxidize the substrate IMP, generating the products xanthosine monophosphate (XMP) and NADH. Known IMPDH inhibitors such as mycophenolic acid (MPA) bind in an IMP & NAD-dependent fashion to IMPDH (Fleming et al, Biochemistry, 35:6990-7 (1996)), and function-based screening of IMPDH must be performed in the presence of IMP and NAD. However, affinity-based screening of IMPDH in the presence of IMP but in the absence of NAD+ yielded novel ligands that bind IMPDH in an IMP-dependent and NAD-independent fashion. These ligands act as functional inhibitors of IMPDH in biochemical assays that follow the production of fluorescent NADH, with IC50s ranging from 50 nM to 10 uM, and in cellular assays such as the inhibition of PBMC cell proliferation upon PHA stimulation with EC50s ranging from 500 nM-50 uM.

### Example 3

### Screening of the Basal, Unactivated Form of a Target Kinase to Identify Ligands that Inhibit Enzymatic Activity

The human kinase p38 is known to play an important role in inflammation, and thus inhibitors of p38 activity are candidates for anti-inflammatory drug discovery (REF). For full activity, the basal form of p38 kinase must be activated by phosphorylation at amino acid residues Thr180 &Tyr182, by MKK6/MEK6Wilson et al., (J. Biol. Chem., 271:27696-27700 (1996)). The phosphorylation of p38 shifts the preferred equilibrium conformation of the DFG loop from the low activity “DFG-out” conformation” to the high activity “DFG-in” conformation (Knighton et al., Science 253:407-414 (1997), Yamaguchi et al., Nature 384:484-489 (1996)). Function-based screening of p38, in the presence of substrate and ATP, has typically utilized the activated form of p38 to ensure a robust & reliable functional read-out. However, affinity-based screening of basal, unphosphorlyated p38 kinase in the absence of ATP and substrate peptide yielded novel ligands that bind to and stabilize the DFG-out, inactive conformation of p38 kinase. These ligands act as highly selective, functional inhibitors of p38 in biochemical assays that report the transfer of radiolabeled ATP to MAPKAPK-2 substrate peptide, with IC50s ranging from 50 nM to 10 uM, and in THP.1 whole cell assays that report the inhibition of LPS-mediated release of TNFalpha, with EC50s ranging from 500 nM-50 uM. Mechanistically such ligands can inhibit activity by preventing activation (phosphorylation), preventing the binding of substrate and/or preventing the binding of ATP.

### Example 4

### Screening of a Target Enzyme in the Absence of a Required Protein Partner to Identify Ligands that Inhibit Enzymatic Activity

The human cyclin-dependent kinase 2 (CDK2) is known to play an important role in cancer cell proliferation, and thus inhibitors of CDK2 activity are candidates for anti-cancer drug discovery (Knockaert et al, TIPS, 23:417-425 (2002)). For full activity, the basal form of CDK2 kinase must bind in a 1:1 stoichiometry with a protein cyclin partner (Knockaert et al, TIPS, 23:417-425 (2002)). Two known cyclins that bind to CDK2 and form active CDK2-cyclin complexes are cyclinA and cyclinE. The binding of cyclin to CDK2 is required for kinase activity, and thus function-based screening of CDK2, in the presence of substrate peptide and ATP, must utilize the cyclin-CDK2 complex to obtain a functional read-out. However, affinity-based screening of basal, unphosphorlyated CDK2 in the absence of cyclin, and in the absence of ATP and substrate peptide, yielded novel ligands that bind selectively to the cyclin-free, inactive form of CDK2. These ligands act as functional inhibitors of CDK2 in conventional cellular proliferation assays using cancer cell lines, with EC50s ranging from 500 nM-50 uM. Mechanistically such ligands can inhibit activity by preventing the binding of cyclin to CDK2, preventing activation (phosphorylation), preventing the binding of substrate, and/or preventing the binding of ATP.

### Example 5

### Screening of the Unliganded form of a Target NHR to Identify Ligands that Inhibit Receptor Activity

The human nuclear hormone receptor (NHR) liver X receptor beta (LXRbeta) is known to play an important role in lipid homeostasis, and thus modulators of LXRbeta activity are candidates for dyslipidemia and cardiovascular drug discovery. For full activity as a transcriptional activator, the basal form of LXRbeta must be activated by binding to a small molecule agonist in a 1:1 stoichiometry. 24-hydroxycholesterol is one naturally occurring small molecule agonist for LXRbeta. (REFLehmann et al., J. Biol. Chem, 272:3137-40 (1997). To identify antagonists or inverse agonists of LXRbeta, a function-based screen must be performed in the presence of agonist to obtain a robust functional read-out. However, affinity-based screening of basal, unliganded LXRbeta in the absence of 24-hydroxycholesterol yielded novel ligands that bind to the agonist-free, basal form of LXRbeta. These ligands act as inverse agonists and antagonists of LXRbeta in whole cell reporter cellular assays (where the LXRbeta activity drives expression of a standard protein reporter such as luciferase) and in whole cell assays that measure the transcription of naturally occurring LXRbeta-dependent transcripts, with EC50s ranging from 50 nM-50 uM. Mechanistically such ligands can inhibit activity by stabilizing an inactive conformation of LXRbeta (inverse agonism) and also by preventing the binding of the naturally occurring agonist(s) to LXRbeta (antagonism).

### Example 6

### Screening of the Unliganded form of a Target GPCR to Identify Ligands that Inhibit Receptor Activity

The human G-protein coupled-receptor (GPCR) m2 muscarinic acetylcholine receptor (m2R) is known to play an important role in cardiovascular processes & schizophrenia, and thus modulators of m2R activity are candidates for cardiovascular & CNS disorder drug discovery (Brown, J. H. & P. Taylor (1996) Muscarinic Receptor Agonists and Antagonists. In The Pharmacological Basis of Therapeutics (Hardman, J. G., et al., eds.) Ninth edition. New York, N.Y.: McGraw-Hill). For full activity, the basal form of m2R must be activated by the binding of a small molecule agonist, and acetylcholine is one naturally occurring small molecule agonist for m2R (Ashkenazi, A. & E. G. Peralta (1994) Muscarinic Acetylcholine Receptors. In Handbook of Receptors and Channels (S. J. Peroutka, ed.) Volume 1. Boca Raton, Fla.: CRC Press). To identify antagonists or inverse agonists of m2R, a function-based screen must be performed in the presence of agonist to obtain a robust functional read-out. However, affinity-based screening of basal, unliganded m2R in the absence of acetylcholine yielded novel ligands that bind to the agonist-free, basal form of m2R. These ligands act as inverse agonists and antagonists of m2R in whole cell assays that measure the cellular responses to added acetylcholine, with EC50s ranging from 100 nM-50 uM. Mechanistically such ligands can inhibit activity by stabilizing an inactive conformation of m2R (i.e., inverse agonism) and also by preventing the binding of the naturally occurring agonist(s) to m2R (i.e., antagonism).

### Example 7

### Screening of the Pro-Enzyme Form of a Target Protease to Identify Ligands that Inhibit Protease Activity

In the blood coagulation process, a cascade of protease activities is initiated to catalyze the formation of a clot. Inhibitors of such proteases can be anti-coagulants. In this cascade, one protease activates its “downstream” protease by enzymatic cleavage, resulting in conversion of the low activity pro-enzyme, or zymogen, form of the downstream protease to the fully activated form (Davie et al, Biochemistry, 30:10363-10370 (1991)). Enzymatic cleavage typically results in an internal splice or the liberation of a peptide fragment from the pro-enzyme. Function-based screening has employed the protealyzed, high activity form of coagulation proteases for inhibitor discovery. However, affinity-based screening of the low activity, or zymogen, form of a coagulation protease can yield novel ligands that bind to the inactive form of the protease. These ligands can act as functional inhibitors of the protease by preventing proteolytic activation (e.g. processing by the upstream protease) in a blood clotting assay or by preventing the binding of the substrate peptide (either by direct competition or allosteric inhibition) in an amidolytic chromogenic assay.

### Example 8

### Screening of an Inactive, Mutant Form of a Target Protease to Identify Ligands that Inhibit Protease Activity

The human protease Factor VIIa (fVIIa) is a critical component of the blood coagulation cascade, and inhibitors of the fvIIa protease can be anti-coagulants. Function-based screening has employed the wild-type, high activity form of fVIIa in complex with soluble Tissue Factor (sTF) for inhibitor discovery. However, affinity-based screening of fVIIa/sTF can been performed with an inactive mutant form of fVIIa protease. The inactive mutant prevents auto-proteolysis during the binding reaction of the affinity screening process, but it still presents almost all of the critical active site residues for ligand binding. Affinity screening of inactive, mutant fVIIa/sTF complex yielded novel ligands that bind to the inactive form of the protease. These ligands act as functional inhibitors of the wild-type, high activity protease in the amidolytic chromogenic assay with IC50s ranging from 1-50 uM.

### Example 9

### Identifying Ligands as Kinase Inhibitors

The invention can be applied to screening unactivated kinases for inhibitors of these kinases. Screening unactivated kinases surprisingly provides more selective inhibitors, in contrast to conventional screening methods relying on functional biochemical readouts that require the use of activated kinases. Activated kinases predominantly exist in what is referred to as the DFG-in conformation while unactivated kinases exist predominantly in the DFG-out conformation. The invention is also based, in part, on the finding that when the DFG is present in the DFG-out conformation, an allosteric binding pocket (e.g., a concave pocket) distinct from the ATP binding site is formed in the unactivated kinase and when this allosteric binding pocket is bound (e.g., by a ligand, e.g., an inhibitor), ATP is indirectly prevented from binding to the ATP site on the kinase. In the absence of ATP and substrate, where the kinase is unactivated and therefore unable to be screened by conventional activity-based assays, there was the surprising finding that the kinase was preferentially in the DFG-out conformation. Because the structural features of the allosteric binding site are more structurally diverse than most ATP binding sites, binders to this new allosteric binding pocket (e.g., concave pocket) are typically found to be more selective kinase inhibitors.

The invention can be applied to methods for identifying new inhibitor binding sites, and for designing inhibitors of kinases, and compositions that include these inhibitors. A method of identifying an inhibitor of a test kinase involves using the 3-dimensional structure of an unactivated reference kinase, e.g., a ser/thr kinase or a tyrosine kinase, e.g., p38 MAPK or c-abl bound to its inhibitor, e.g., BIRB 796 or STI-571 or variant thereof (i.e., Gleevec® or variant thereof), respectively. Based on the location of the DFG (e.g., Asp-Phe/Leu/W-Gly, e.g., DFG, DLG, or DWG) motif of the reference kinase, the DFG motif in an unactivated test kinase can be located (e.g., by multiple sequence alignment and/or overlay of the test kinase onto the structure of the reference kinase). An example of a reference kinase is unactivated insulin receptor kinase (1irk). In 1irk, the DFG motif corresponds to Asp1150-Phe1151-Gly1152. Another example of a reference kinase is c-abl. The DFG motif of 1fpu (PDB accession code for c-abl bound to an STI-571 variant) or 1iep (PDB accession code for c-abl bound to STI-571) includes Asp381-Phe382-Gly383. The middle residue, phenylalanine can sometimes be tryptophan (Trp, W) or leucine (Leu, L). Thus the DFG motif can have the amino acid sequences DFG, DWG, or DLG. Test kinases (e.g., unactivated test kinases) can be aligned with a reference kinase (e.g., an unactivated reference kinase), for which the location of the DFG is known (e.g., p38 MAPK or c-abl bound to its respective inhibitors BIRB 796 and an STI-571 variant) in a multiple sequence alignment to determine the position of the DFG motif in the test kinases. Multiple sequence alignments can be accomplished using, for example, CLUSTALW, FASTA, or HMMER.

The helix α-C of a reference kinase(s) (e.g., an unactivated reference kinase) can be used to locate the helix α-C of a test kinase (e.g., an unactivated test kinase). For example, in 11RK, the alpha helix which includes Val1050-Met1051 corresponds to the helix α-C. In another example, the helix α-C of 1IEP includes Val289-Met290. A multiple sequence alignment of sequences for which the location of the helix α-C is known (e.g., 3-dimensional structure has been solved experimentally) can be used to identify the location of the helix α-C in other sequences whose 3-dimensional structure has not yet been determined experimentally.

Once the DFG motif is located, one can determine if the DFG motif is in the out conformation (DFG-out). For kinases that are in a fully active state, the activation loop is often found in an extended or open conformation and the DFG motif is usually found in the DFG-in conformation (Knighton et al., Science 253:407-414 (1997), Yamaguchi et al., Nature 384:484-489 (1996)). The new methods described herein are for identifying the DFG motif in a DFG-out conformation. To determine if the DFG motif of the test kinase is in the DFG-out conformation, the distance between the DFG motif and the helix α-C of the test kinase is measured. Specifically, the shortest distance between the following two atom sets: 1) non-backbone heavy atoms of the Phe/Leu/Trp residue (the center residue) of the DFG motif and 2) alpha carbons in any residue in helix α-C, is measured. A distance of greater than or equal to 11 Å, and less than or equal to 20 Å (e.g., 11.0, 11.2, 11.4, 11.6, 11.8, 11.9, 12, 13, 14, 15, 16, 17, 18, 19, or 20 Å) defines a DFG motif as being in the DFG-out conformation. For example, 1iep represents c-abl bound to its inhibitor, an STI-571 variant. In 1iep, the shortest distance between the non-backbone heavy atoms of Phe and an alpha carbon of its helix α-C was measured to be 11.9 Å and thus the DFG motif for 1iep is designated as DFG-out. In rare cases, a reference kinase can be found in the DFG-out conformation but be in the activated state. Using an activated reference kinase in the DFG-out conformation can still be useful in identifying kinase inhibitors since the DFG-out conformation may still form the allosteric pocket to which an inhibitor can bind.

When the DFG motif is in the DFG-out position, it can induce the formation of a concave pocket (e.g., allosteric binding site), wherein the surface of the concave pocket is formed in part by amino acids designated herein as X through Y. The concave pocket (e.g., the allosteric binding site), to which an inhibitor can be designed to bind, can be localized by the method of alignment. In one embodiment, localizing the concave pocket for kinase inhibitor binding (e.g., test kinase inhibitor binding) can involve aligning the amino acid sequence X through Y of a test kinase with the sequence X through Y of one or more reference kinases some of which are listed in Table 1. In another embodiment, X through Y can consist of Leu104 through Ala111 of a protein whose PDB accession code is 1kv1 (chain A) as determined by sequence alignment analysis (see Table 1, below). In another embodiment, the reference kinase amino acids X through Y can consist of Leu 104 through Ala111 of a protein whose PDB accession code is 1kv2 (chain A) as determined by sequence alignment analysis. In another embodiment, test kinase amino acids X through Y consist of amino acids analogous to Leu104 through Ala111 of a protein whose PDB accession code is 1kv1 (chain A) or 1kv2 (chain A); Ile313 through Asn322 of 1iep (chain A), 1iep (chain B), 1fpu (chain A) or 1fpu (chain B), and/or Leu1073 through Ala1080 of 1irk as determined by sequence alignment analysis (e.g., using the Smith-Waterman algorithm for protein sequence alignment (e.g., as described in Smith et al., 1981, J Mol Biol 147:195-197 and Pearson, 1991, Genomics 11:635-650).

By “amino acids analogous to” it is meant that amino acid residues are similar but not identical. Thus, amino acid residues having similar side chains can be said to be analogous and have been defined so in the art. These similarities may allow the amino acids to act “analogously” within the three-dimensional structure of the kinase (e.g., similar in their interactions with other amino acids within 3-dimensional proximity). These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, for example, analogous amino acids to lysine would be arginine or histidine. As another example, aspartic acid is analogous to glutamic acid as described herein. Thus, the region of a test kinase which aligns (e.g., using the Smith-Waterman algorithm for protein sequence alignment) with the amino acid sequence X through Y (e.g., see Table 1) of the reference kinase, including gaps, is the homologous region which partially forms the concave binding pocket induced by the DFG in the DFG-out conformation. By identifying the homologous test kinase amino acids and thus the test kinase concave pocket induced by DFG-out, the allosteric binding site is identified and an inhibitor can be designed to bind to the test kinase at this site. Within this homologous site, there can be certain amino acids which are analogous between the test kinase and the reference kinase.

Once the “homologous” amino acids are determined in the unactivated test kinase, they can be used to design a scaffold with the correct topology (shape) and electronic properties to fit the allosteric binding site (e.g., concave pocket). A library (e.g., a mass-coded library, see U.S. Pat. Nos. 6,207,861 and 6,147,344) can be provided or is then synthesized based on that scaffold. Allosteric ligands (e.g., inhibitors) are identified by affinity screening of the library (e.g., a provided mass-coded library or a designed mass-coded library) with the unactivated test kinase under conditions where the allosteric site (e.g., present when the DFG motif is in the DFG-out conformation) predominates (e.g., the test kinase is unactivated in the absence of ATP and substrate).

In another aspect, the library (e.g., mass-coded library) can be screened against the activated form of a test kinase in the instance where an ATP site binder is desired. In this case, library screening occurs under conditions in which the test kinase is in the activated state and is competent to bind ATP and/or substrate. Optionally, screening can be conducted in the presence or ATP and/or substrate. In one instance, the library can be provided. In another instance, the library can be designed based on the topology or electronic properties of the allosteric site formed by the test kinase being in the DFG-out conformation. In either case, the library members can be mass-coded after being synthesized.

TABLE 1

| Reference kinases* | Region X through Y |
|---|---|
| 1kv1 (chain A) | Leu104-Ala111 |
| 1kv2 (chain A) | Leu104-Ala111 |
| 1iep (chain A) | Ile313-Asn322 |
| 1iep (chain B) | Ile313-Asn322 |
| 1fpu (chain A) | Ile313-Asn322 |
| 1fpu (chain B) | Ile313-Asn322 |
| 1irk | Leu1073-Ala1080 |

*PDB accession code

Because conventional screening methods typically employ functional assays that require activated kinases predominantly in the DFG-in conformation, inhibitors identified by these screens largely target the ATP binding site and are thus ATP competitive. Kinases with inhibitors bound in this fashion exhibit a DFG-in conformation and inhibitors of this type frequently have the disadvantages of less selectivity and increased side effects and toxicity. Conventional methods of designing kinase inhibitors or binders to kinases have relied on the kinase being in the active state with a DFG-in conformation. In contrast, the methods described herein have the advantage of allowing the design of inhibitors to unactivated kinases with the DFG-out conformation and provide inhibitors with greater selectivity. The methods herein allow for the identification of inhibitors of kinases without relying on the kinase being in the active state, though the methods can be applied to a kinase in the active state if desired.

In the DFG-in conformation, the center residue (Phe, Leu, or Trp) is buried in a hydrophobic pocket in the groove between the two lobes of the kinase (Frantz, 2002, Nature Reviews 1:253). When the DFG motif is in the DFG-out conformation, however, one face of the side chain of the center residue (Phe, Leu, or Trp) helps to shield the inhibitor while the other face is exposed to solvent. This center residue, is also important in binding the divalent ion which, in most cases, is required by kinases for activity. The center residue coordinates the position of the aspartate, the first residue of the DFG motif. This aspartate is one of the ligands of the coordination sphere of a magnesium (or manganese) ion. This divalent ion (magnesium or manganese) in turn coordinates the beta and gamma phosphates of ATP thus supporting ATP binding. It should be noted that a divalent ion is not absolutely required for DFG interactions with ATP phosphates. This conformation exposes a large hydrophobic pocket in the kinase that is spatially distinct from the ATP-binding pocket. When bound to this large hydrophobic pocket or allosteric site an inhibitor locks the DFG motif in the DFG-out conformation. The physical positioning of DFG in this conformation (e.g., DFG-out) is what prevents ATP binding, which in turn inhibits kinase function.

Mixtures of compounds (e.g., libraries of small molecule compounds, e.g., mass-coded combinatorial libraries) can be screened (e.g., by computational modeling, or by affinity screening) for those that can bind the test kinase (e.g., the unactivated test kinase, e.g., in the absence of ATP and/or substrate). In one embodiment, a mass-coded library can be designed from a scaffold determined by the topology and electrostatic properties of the new allosteric binding site (e.g., concave pocket) formed in a kinase when its DFG is in the DFG-out conformation. In another embodiment, a mass-coded library can be provided and screened for kinase binders that inhibit or modulate kinase activity. In yet another embodiment, individual discrete compounds can be screened by the methods of this invention by first creating mass-coded mixtures of the compounds by calculating the molecular weight of each compound and then mixing appropriate compounds together. Compounds that bind the allosteric site (e.g., the concave pocket) can also be designed by computer modeling based on the allosteric site (e.g., concave binding pocket) identified by the methods described herein and the potential binders can then be synthesized based on this information.

U.S. Pat. Nos. 6,207,861 and 6,147,344 (see supra) describe useful methods of screening libraries of compounds (e.g., mass-coded libraries designed based on a scaffold that fits the new allosteric binding site) which can be applied to methods described herein. They can be applied to designing compound mixtures based on the allosteric site or based on a scaffold targeting the allosteric site.

Target Inhibitor Synthesis

Target (e.g., kinase) inhibitors can be synthesized by methods well established in the organic synthesis literature (Gazit et al., 1989, J. Med. Chem.32:2344-2352; McKenna et al., 2002, J. Med. Chem.45:2173-2184; Levitzki, 2002, Eur. J. Cancer 38,Suppl 5:S11-8). Moreover, methods for synthesizing mixtures of compounds comprising mass-coded libraries have also been described (Shipps, et al., 1997, Proc. Natl. Acad. Sci USA 94:11833-11838; Shipps et al., 1996, Bioorg. Med. Chem. 4:655-657; Makara et al., 2002, Org. Lett. 4:1751-1754; Makara, 2001, J. Org. Chem.66:5783-5789). Synthetic chemistry transformations and protecting group methodologies (protection and deprotection) useful in synthesizing the inhibitor compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The inhibitors described herein can contain one or more asymmetric centers and thus occur as racemates and racemic mixtures, single enantiomers, individual diastereomers and diastereomeric mixtures. All such isomeric forms of these compounds are expressly included in the present invention. The inhibitors described herein can also be represented in multiple tautomeric forms, all of which are included herein. The inhibitors can also occur in cis-or trans-or E-or Z-double bond isomeric forms. All such isomeric forms of such inhibitors are expressly included in the present invention.

Inhibitors can be identified or selected by screening a mixture of compounds (e.g., a library of compounds, e.g., a combinatorial library, e.g., mass-coded combinatorial library) with a method such as ALIS (described above) which is a method of affinity screening that can identify allosteric ligands of a kinase in the unactivated state. Inhibitors can also be identified or selected by screening a mixture of compounds by using conventional library screening methods known in the art.

Target Inhibition Assays

The potential inhibitor selected, identified, or designed by the aforementioned process can be assayed to determine its ability to inhibit a test target and/or a signaling pathway that depends on the target. The assay can be in vitro or in vivo. Inhibition can be measured by various methods, including, for example, Phosphorylation assays, Scintillation Proximity Assay (Amersham), DELFIA assay (Perkin Elmer), or a continuous spectrophotometric assay, as well as cellular functional and tumor cell assays (Spencer-Fry, J., et al., 1997, Journal of Biomolecular Screening 2(1):25-32; Braunwalder et al., 1996, Anal. Biochem. 238; 159-64; Barker et al., 1995, Biochemistry 34:14843-51).

Traditional methods known in the art can also be used to confirm kinase inhibition. These methods involve the addition of radiolabeled ATP and substrate to a reaction mix consisting of the kinase to be inhibited, the potential inhibitor, and ingredients which provide conditions similar to that found physiologically. Measurement of the incorporation of radiolabeled phosphate into the substrate relative to control can then provide information which identifies the potential inhibitor as a confirmed kinase inhibitor.

For all potential inhibitors and inhibitors confirmed by assays described herein, further refinements to the structure of the potential inhibitor to improve affinity, inhibitory activity, and/or in vivo properties will generally be necessary. This can be accomplished by standard techniques employed in medicinal chemistry and can be made by successive iterations of any/or all of the steps provided by the inhibitor screening assays described herein.

Uses for Target Inhibitors

The ligands discovered by the methods described herein can be useful for inhibition of a target which is activatable. For example, the ligands can be inhibitors of G-protein coupled receptors, phosphatases, transferases, synthases, kinases, proteases, nuclear hormone receptors, dimerizing receptors, transporters, isomerases, polymerases, protein-protein domains, transcription factors, hydrolases, and membrane-associated proteins and enzymes. To the extent that a target is associated with disease, the ligands discovered by the methods described herein can be formulated into a pharmaceutical composition for the diagnosis and/or treatment of such disease in a mammal, for example in a human. Such disease may include, cancer, inflammation, neurological disorders, obesity, senescence, viral infections, bacterial infections, and ailments associated with the attack of biological warfare. The ligands discovered by the methods described herein are also useful in inhibiting biological activity of any target comprising greater than 90%, alternatively greater than 85%, or alternatively greater than 70% sequence homology with a target sequence. The inhibitors described herein are also useful for inhibiting the biological activity of any target (e.g., enzyme, e.g., kinase) comprising a subsequence, or variant thereof, of any target (e.g., enzyme, e.g., kinase) that comprises greater than 90%, alternatively greater than 85%, or alternatively greater than 70% sequence homology with a kinase subsequence, including subsequences of the kinases mentioned herein. Such subsequence preferably comprises greater than 90%, alternatively greater than 85%, or alternatively greater than 70% sequence homology with the sequence of an active site or subdomain of an enzyme (e.g., a kinase). The subsequences, or variants thereof, comprise at least about 250 amino acids, or alternatively at least about 120 amino acids.

Uses for Kinase Inhibitors

The inhibitors identified by the methods described herein can be useful for inhibition of kinase activity of one or more enzymes. Specifically, the compounds described herein are useful as inhibitors of tyrosine, serine/threonine, lipid or histidine kinases. Examples of kinases (e.g., test kinases) that are inhibited by the compounds and compositions described herein and against which the methods described herein are useful, include, but are not limited to serine kinases, threonine kinases, tyrosine kinases, and/or lipid kinases. Specific examples of potential test kinases for which an inhibitor can be designed are listed in Table 2. These kinases can be screened in both the basal and activated states. The inhibitors identified by the methods described herein are suitable for use in the treatment of diseases and disease symptoms that involve one or more of the aforementioned protein kinases. In one embodiment, the inhibitors identified by the methods described herein are particularly suited for inhibition of or treatment of disease or disease symptoms mediated by kinases.

The inhibitors described herein are also useful for inhibiting the biological activity of any enzyme (e.g., kinase), comprising greater than 90%, alternatively greater than 85%, or alternatively greater than 70% sequence homology with a kinase sequence, including the kinases mentioned herein. The inhibitors described herein are also useful for inhibiting the biological activity of any enzyme (e.g., kinase) comprising a subsequence, or variant thereof, of any enzyme (e.g., kinase) that comprises greater than 90%, alternatively greater than 85%, or alternatively greater than 70% sequence homology with a kinase subsequence, including subsequences of the kinases mentioned herein. Such subsequence preferably comprises greater than 90%, alternatively greater than 85%, or alternatively greater than 70% sequence homology with the sequence of an active site or subdomain of an enzyme (e.g., a kinase). The subsequences, or variants thereof, comprise at least about 250 amino acids, or alternatively at least about 120 amino acids.

The inhibitors described herein are useful in inhibiting kinase activity. As such, the compounds, compositions and methods of this invention are useful in treating kinase-mediated disease or disease symptoms in a mammal, particularly a human. Kinase mediated diseases are those wherein a protein kinase is involved in signaling, mediation, modulation, or regulation of the disease process. Kinase mediated diseases are exemplified by, but are not limited to, the following disease classes: cancer, inflammation, neurological disorders, and obesity.

TABLE 2

| | Target | Swiss Prot |
|---|---|---|
| 1 | Akt (activated) | P31749 |
| 2 | Akt (basal) | P31749 |
| 3 | A-Raf | P10398 |
| 4 | ATR | Q13535 |
| 5 | Bcr-Abl | P00519 |
| 6 | BLK | P51451 |
| 7 | B-Raf | P15056 |
| 8 | Btk | Q06187 |
| 9 | CDK2 | P24941 |
| 10 | CDK4 | P11802 |
| 11 | CDK6 | Q00534 |
| 12 | C-met | P08581 |
| 13 | ERK1 | P27361 |
| 14 | ERK2 | P28482 |
| 15 | FAK | Q05397 |
| 16 | FGFR | P11362 |
| 17 | Flt3 | P36888 |
| 18 | IGF1RK | P08069 |
| 19 | IKK1 | O15111 |
| 20 | ILK-1 | Q13418 |
| 21 | IRAK4 | Q8TDF7 |
| 22 | Itk | Q08881 |
| 23 | JNK1 | P45983 |
| 24 | Jnk-2 | P45984 |
| 25 | Jnk3 | P53779 |
| 26 | Lck | P06239 |
| 27 | MAPKAPK-2 | P49137 |
| 28 | MEK1 | Q02750 |
| 29 | MSK1 | O75582 |
| 30 | p38 MAPK | Q16539 |
| 31 | PAK | Q13153 |
| 32 | PDGR | P09619 |
| 33 | PDGS | P16234 |
| 34 | PDK1 | Q15118 |
| 35 | Pl-3K alpha | P42336 |
| 36 | Pl-3K gamma | P48736 |
| 37 | Pim-1 | P11309 |
| 38 | Pim-2 | Q9P1W9 |
| 39 | PKC alpha | P17252 |
| 40 | PkC beta | P05771 |
| 41 | PKC gamma | P05129 |
| 42 | PKC theta | Q04759 |
| 43 | PLK1 | P53350 |
| 44 | PRAK | O60491 |
| 45 | Raf1 (C-Raf) | P04049 |
| 46 | RAFTK | Q14289 |
| 47 | RET | P07949 |
| 48 | Rlk/Txk | P42681 |
| 49 | ROCK | Q13464 |
| 50 | RSK | Q15418 |
| 51 | Sphingosine Kinase | Q9NYA1 |
| 52 | SRC | P12931 |
| 53 | Syk | P43405 |
| 54 | Tak1 | O43318 |
| 55 | TGF-β R1K | P36897 |
| 56 | Tpl-2/COT | P41279 |
| 57 | TrkA | P04629 |
| 58 | VEGFR3 | P35916 |
| 59 | Zap-70 (activated) | P43403 |
| 60 | ZAP-70 (basal) | P43403 |

All references cited herein, whether in print, electronic, computer readable storage media or other form, are expressly incorporated by reference in their entirety, including but not limited to, abstracts, articles, journals, publications, texts, treatises, internet web sites, databases, software packages, patents, and patent publications. A number of embodiments of the invention have been described. Nevertheless, it will be understood that various modifications may be made without departing from the spirit and scope of the invention. Accordingly, other embodiments are within the scope of the following claims and the Summary (above).

1. A method for screening a mixture of compounds against an unactivated form of a target moiety, the method comprising
a. providing a mixture of compounds;
b. incubating the mixture with the unactivated form of the target moiety to form compound:target complexes;
c. separating compound:target complexes from unbound compounds and targets; and
d. dissociating compound:target complexes;
e. identifying the dissociated compounds which had bound to the target by passing the compound through a mass spectrometer, wherein the identified compounds bind to the target moiety with an affinity of Kd between 1 pM and 50 μM.

2. A method for screening a mixture comprising of compounds against a mixture of different forms of a target moiety, the method comprising
a. providing a mixture of compounds;
b. providing a mixture of ligand-bound forms and ligand-free forms of a target moiety;
c. incubating the mixture of compounds with the mixture of the target moiety to form compound:target complexes;
d. separating compound:target complexes from unbound compounds and target moieties;
e. dissociating compound:target complexes; wherein the dissociated compounds separated from the target are designated new ligands;
f. identifying new ligands from among the compounds present in compound:target complexes bound to any form of the target moiety by passing compound:target complexes through a mass spectrometer to identify ligands bound to any form of the target moiety, wherein the identified ligands bind to the target moiety with an affinity of Kd between 1 pM and 50 μM; and
g. incubating the new ligands identified in step f with ligand-bound and ligand-free forms of the target moiety, wherein steps e to f are repeated to delineate which compounds bind to the ligand-bound form versus which compounds bind to the ligand-free form of the target moiety.

3. The method of claim 1, wherein the compound mixture is mass-coded to ensure at least 90% of the compounds having a unique ion mass detectable by a mass spectrometer.

4. The method of claim 2, wherein the compound mixture is mass-coded to ensure at least 90% of the compounds having an unique ion mass detectable by a mass spectrometer.

5. The method of claim 2, wherein the mixture of target forms comprises unactivated forms, inactive forms, and active forms.

6. The method of claim 2, wherein the mixture of target forms comprises monomeric forms and multimeric forms.

7. The method of claim 2, wherein the mixture of target forms comprises ligand-bound forms and ligand-free forms.

8. The method of claim 2, wherein the mixture of target forms comprises cofactor-bound forms and cofactor-free forms.

9. The method of claim 3 or 4, wherein the mixture of target forms comprises unactivated forms and active forms.

10. The method of claim 3 or 4, wherein the mixture of target forms comprises monomeric forms and multimeric forms.

11. The method of 3 or 4, wherein the mixture of target forms comprises ligand-bound forms and ligand-free forms.

12. The method of 3 or 4, wherein the mixture of target forms comprises cofactor-bound forms and cofactor-free forms.

13. A method for discovering a test kinase inhibitor, the method comprising:
a) incubating the test kinase with a mass-coded library of compounds in the absence of ATP and/or peptide substrate;
b) separating bound compounds from unbound compounds;
c) identifying the bound compounds by mass-spectrometry; and
d) demonstrating test kinase inhibitory activity of the bound compound in a kinase inhibition assay.

14. The method of claim 13, wherein the test kinase is a full-length kinase.

15. The method of claim 13, wherein the test kinase comprises a truncated fragment of the full-length kinase that contains a catalytic domain.

16. The method of claim 13, wherein the kinase is a kinase variant or mutant.

17. The method of claim 13, wherein separating bound compounds from unbound compounds is by size exclusion chromatography.

18. The method of claim 13, wherein mass-spectrometry is done by comparing to a database of mass-coded compounds.

19. The method of claim 13, wherein the test kinase is unactivated.

20. The method of claim 13, wherein the test kinase is in a basal form exhibiting low catalytic activity.

21. The method of claim 13, wherein the test kinase is activated.

22. The method of claim 14, wherein the test kinase is partially active relative to its physiologically active state.

23. A method of designing an inhibitor, the method comprising
a) comparing an unactivated test kinase to an Inactivated reference kinase whose 3-dimensional structure is known;
b) identifying an allosteric binding site; and
c) designing an inhibitor based on the allosteric binding site.

24. The method of claim 23, wherein step c) comprises the steps
c1) designing a scaffold based on topological and electronic properties of the allosteric binding site; and
c2) designing a mass-coded library based on the scaffold.

25. The method of claim 23, wherein step c) comprises providing a mass-coded library.

26. The method of claim 24, further comprising step d) screening the mass-coded library for compounds that bind to the test kinase; and e) determining whether the kinase binder inhibits the test kinase, thereby designing an inhibitor of the test kinase.

27. The method of claim 25, wherein screening comprises affinity screening with the test kinase.

28. The method of claim 23, wherein the reference kinase is selected from the group consisting of kinases with Protein Data Bank identifiers lkyl chain a and 1kv2 chain a.

29. The method of claim 23, wherein the reference is selected from the group consisting of kinases with Protein Data Bank identifiers 1iep chain a, 1iep chain b, 1fpu chain a, and 1fpu chain b.

30. The method of claim 23, wherein the reference kinase has a Protein Data Bank identifier 1irk.

31. The method of claim 23, wherein the reference is selected from the group consisting of kinases with Protein Data Bank identifiers 1g3n chain a and 1g3n chain b.

32. The method of claim 23, wherein the allosteric binding site is spatially distinct from the ATP binding site and the activation loop.

33. The method of claim 23, wherein the allosteric binding site is identified by locating a DFG motif.

34. The method of claim 33, wherein the DFG motif comprises a DWG or a DLG sequence.

35. The method of claim 33, wherein the DFG motif is greater than 11 Å and less than 20 Å in distance from an helix alpha-C, and the DFG motif is in the DFG-out conformation.

36. The method of claim 35, wherein the helix alpha-C is homologous in relative 3-dimensional location to insulin receptor kinase helix alpha C containing Val1050-Met1051.

37. The method of claim 35, wherein the helix alpha-C is homologous in relative 3-dimensional location to c-abl helix alpha-C containing Val289-Met290.

38. The method of claim 23, further comprising determining whether the potential binder inhibits the test kinase.

39. A method of designing an inhibitor of a test kinase, the method comprising
a) comparing the test kinase to a reference kinase whose 3 dimensional structure is known;
b) identifying an allosteric binding site;
c) designing a scaffold targeting the allosteric binding site;
d) providing a mixture of compounds based on the scaffold;
e) identifying ligands for the allosteric site by affinity screening against the kinase; and
f) demonstrating kinase inhibitory activity by the ligand in a kinase assay.

40. The method of claim 39, wherein the test kinase and the reference kinase are unactivated.

41. The method of claim 39, wherein the test kinase is a full-length kinase.

42. The method of claim 39, wherein the test kinase comprises a truncated fragment of the full-length kinase that contains a catalytic domain.

43. The method of claim 39, wherein the test kinase is in a basal form exhibiting low catalytic activity.

44. The method of claim 39, wherein the test kinase is activated.

45. The method of claim 39, wherein the test kinase is partially active relative to its physiologically active state.

46. The method of claim 39, wherein step c) comprises designing a scaffold based on topological and electronic properties of the allosteric binding site.

47. The method of claim 39, wherein the mixture of compounds based on the scaffold consists of a mass-coded library of potential kinase ligands.

48. The method of claim 39, wherein step e) comprises the steps of
e1) incubating the test kinase with the mass-coded library to allow ligands of the library to bind the test kinase;
e2) separating kinase-bound ligands from unbound ligands;
e3) separating the kinase from the bound ligand; and
e4) identifying bound compounds by mass-spectrometry;

49. A method of designing an inhibitor, the method comprising
a) comparing a test kinase to a reference kinase whose 3-dimensional structure is known;
b) identifying an allosteric binding site;
c) designing a scaffold targeting the allosteric binding site; and
d) providing mixtures of compounds based on the scaffold.

50. The method of claim 49, the method further comprising
e) identifying ligands for the allosteric site by affinity screens against the kinase.

51. The method of claim 50, the method further comprising
f) demonstrating kinase inhibitory activity.

52. A method of designing an inhibitor of a test kinase, the method comprising
a) using a 3-dimensional structure of a reference kinase to locate a DFG motif in the test kinase;
b) identifying an allosteric binding site formed in the test kinase by the DFG motif in the DFG-out conformation, wherein the allosteric binding site is spatially distinguishable from an ATP binding site and an activation loop;
c) designing a scaffold based on the allosteric binding site;
d) providing a mixture of compounds based on the scaffold;
e) screening the mixture of compounds for kinase binders;
f) separating kinase binders from non-binders;
g) identifying the kinase binders by mass-spectrometry;
h) optionally contacting the binder to the kinase under conditions and for a time sufficient to allow the binder to bind to the kinase; and
e) demonstrating that the kinase is rendered non-functional or less functional by the binder; thereby identifying an inhibitor of a test kinase.

53. A method of designing an inhibitor of a test kinase, the method comprising
a) using a 3-dimensional structure of a reference kinase to locate a DFG motif in the test kinase;
b) identifying an allosteric binding site formed by the DFG motif in the DFG-out conformation, wherein the allosteric binding site is spatially distinct from an ATP binding site and an activation loop;
c) designing a scaffold based on the allosteric binding site;
d) synthesizing a mixture of compounds based on the scaffold;
e) identifying ligands for the allosteric site by affinity screens of the mixtures of compounds against the kinase; and
f) demonstrating kinase inhibitory activity.

54. The method of claim 41, 42, 49, 52, or 53, wherein the test kinase is unactivated.

55. The method of claim 39, 49, 52, or 53, wherein the reference kinase is selected from the group consisting of kinases with Protein Data Bank identifiers lkyl chain a and 1kv2 chain a.

56. The method of claim 39, 49, 52, or 53, wherein the reference kinase is selected from the group consisting of kinases with Protein Data Bank identifiers 1iep chain a, 1iep chain b, 1fpu chain a, and 1fpu chain b.

57. The method of claim 39, 49, 52, or 53, wherein the reference kinase has a Protein Data Bank identifier 1irk.

58. The method of claim 39, 49, 52, or 53, wherein the reference kinase is selected from the group consisting of kinases with Protein Data Bank identifiers 1g3n chain a and 1g3n chain b.

59. The method of claim 39, 49 or 52, wherein the allosteric binding site is spatially distinct from the ATP binding site and the activation loop.

60. The method of claim 13, 23, 39, 49, 52, or 53, wherein the test kinase is p38 MAP kinase.

61. The method of claim 13, 23, 39, 49, 52, or 53, wherein the test kinase is c-abl.

62. A method of identifying an inhibitor of a test kinase, the method comprising
a) providing a test compound that binds to the test kinase without physically binding to an ATP binding site on the test kinase, wherein the test compound binds to an allosteric site present when a DFG motif of the test kinase is in a DFG-out position;
b) determining if ATP can bind to the ATP binding site on the test kinase, wherein indirectly interfering with the binding of ATP to the test kinase by binding of the test compound to the allosteric site of the test kinase identifies the test compound as a kinase inhibitor; and
c) optionally performing a kinase assay in the presence or absence of the inhibitor bound to the test kinase, wherein decreased kinase activity in the presence of the test compound relative to kinase activity in the absence of the test compound further confirms the test compound is an inhibitor of the test kinase.

63. A method of identifying an inhibitor of a test kinase, the method comprising
a) providing a test compound that binds to the test kinase without physically binding to an ATP binding site on the test kinase; and
b) determining if ATP can bind to the ATP binding site on the test kinase, wherein interfering with the binding of ATP to the test kinase by binding of the test compound to the test kinase identifies an inhibitor of the test kinase, wherein the test compound confines a DFG motif of the test kinase in a DFG-out position.

64. A method of identifying an allosteric binding site spatially distinguishable from an ATP binding site and an activation loop, the method comprising
a) comparing a test kinase to a reference kinase whose 3-dimensional structure is known;
b) locating a DFG motif in the test kinase based on its location in the reference kinase; and
c) measuring shortest distance between an alpha carbon residue in alpha helix C and a non-backbone heavy atom of phenylalanine, leucine or tryptophan of the DFG motif of the test kinase, wherein a distance of greater than 11 Å and less than 20 Å characterizes the DFG motif of the test kinase as in the DFG-out conformation; and
d) identifying amino acids, partially forming a concave pocket formed by the DFG motif in the DFG-out conformation, by locating amino acids in the test kinase analogous to amino acids X through Y of the reference kinase, wherein locating the concave pocket identifies the allosteric binding site.

65. A method of identifying an allosteric binding site, spatially distinct from an ATP binding site and an activation loop, for an inhibitor of a test kinase, the method comprising
a) locating a DFG motif in the test kinase by comparing tertiary structure of the test kinase to tertiary structure of a kinase whose 3-dimensional structure is known;
b) allowing a test compound to bind to the allosteric site when the DFG motif is in an DFG-out position;
c) determining if the test compound inhibits the activity of the test kinase; and
d) determining if the test compound binds the test kinase at an allosteric site formed when the DFG motif is in a DFG-out conformation, wherein binding of the test compound such that the DFG motif is confined in the DFG-out position, identifies the allosteric binding site of the test kinase.

66. The method of any one of claim 33, 52, 53, 64, or 65, wherein locating the DFG motif comprises multiple sequence alignment.

67. The method of claim 65, wherein determining if the test compound inhibits the activity of the test kinase comprises a kinase assay.

68. The method of claim 65, wherein determining if the test compound binds the test kinase at an allosteric site formed when the DFG motif is in the DFG-out conformation comprises labeling the test compound.

69. The method of claim 65, wherein determining if the test compound binds the test kinase at an allosteric site formed when the DFG motif is in the DFG-out conformation comprises producing and analyzing an x-ray crystal of the test compound bound to the kinase.

70. An inhibitor of the method of claim 13, 23, 39, 49, 52, 53, 62, or 63.

71. The inhibitor of claim 70, wherein the inhibitor is not an inhibitor of p38MAPK, c-abl, or insulin receptor kinase.

72. The inhibitor of claim 70, wherein the inhibitor inhibits p38MAPK.

73. The inhibitor of claim 70, wherein the inhibitor inhibits c-abl.

74. The inhibitor of claim 70, wherein the inhibitor inhibits insulin receptor kinase.

75. A pharmaceutical composition comprising the inhibitor of claim 63 or 64.

76. A kit comprising any one of the inhibitors of claim 70.

77. A method of inhibiting kinase activity in a subject comprising the step of administering to the subject a compound comprising the inhibitor of claim 70.

78. The method of claim 77, wherein the subject is a mammal.

79. The method of claim 78, wherein the mammal is a human.

80. A method of treating a kinase-mediated disease or disease symptoms in a subject comprising administration to said subject of a compound comprising the inhibitor of claim 70.

81. The method of claim 80, wherein the subject is a mammal.

82. The method of claim 81, wherein the mammal is a human.

83. A method of treating disease or disease symptoms in a subject comprising the step of administering to said subject of a compound comprising the inhibitor of claim 70.

84. The method of claim 83, wherein the subject is a mammal.

85. The method of claim 84, wherein the mammal is a human.

86. A method of making a pharmaceutically useful composition comprising combining the inhibitor of claim 70 with one or more pharmaceutically acceptable carriers.

87. The inhibitor of claim 70, further comprising combining an additional therapeutic agent.

88. The method of claim 13, 23, 39, 42, 44, 49, 52, or 53, wherein the DFG in the DFG-out position induces formation of a concave pocket, wherein the surface of the concave pocket is formed in part by amino acids X through Y, wherein X is a first amino acid in a contiguous sequence of amino acids and Y is a last amino acid in the contiguous sequence of amino acids that form in part the concave pocket.

89. The method of claim 88, wherein amino acids X through Y consist of Leu 104 through Ala 111 of a protein whose PDB accession code is 1kv2.

90. The method of claim 88, wherein amino acids X through Y consist of amino acids homologous to Leu104 through Ala111, of a protein whose PDB accession code is 1kv2, as determined by sequence alignment analysis.
